SEQUENCE LISTING (87 sequences)

SEQ ID NO: 1 - protein, 28 aa, Artificial Sequence
  FlgII-28
ESTNILQRMRELAVQSRNDSNSATDREA

SEQ ID NO: 2 - protein, 22 aa, Artificial Sequence
  Csp22
AVGTVKWFNAEKGFGFITPDDG

SEQ ID NO: 3 - DNA, 19 bp, Artificial Sequence
  FLS 2.1 Solyc02g070890-F
aatgggaacttggacaaca

SEQ ID NO: 4 - DNA, 23 bp, Artificial Sequence
  FLS 2.1 Solyc02g070890-R
acaccaaagctgaatacatctac

SEQ ID NO: 5 - DNA, 24 bp, Artificial Sequence
  Solyc02g071790-F
atggtttcaatcgataagttgtac

SEQ ID NO: 6 - DNA, 24 bp, Artificial Sequence
  Solyc02g071790-R
ccgacttcttgaggtattctcccg

SEQ ID NO: 7 - DNA, 23 bp, Artificial Sequence
  Solyc02g071800-F
atgggtcttcaatgcatgtgttg

SEQ ID NO: 8 - DNA, 23 bp, Artificial Sequence
  Solyc02g071800-R
taggtccagttcctctagctgag

SEQ ID NO: 9 - DNA, 23 bp, Artificial Sequence
  Solyc02g071810 /  Solyc02g071820-F
tacaccgtgacactgcactttgc

SEQ ID NO: 10 - DNA, 23 bp, Artificial Sequence
  Solyc02g071810 /  Solyc02g071820-R
gggcttgaaattagcttcaaaag

SEQ ID NO: 11 - DNA, 21 bp, Artificial Sequence
  Solyc02g071860-F
atgccaagaatcttcaatttc

SEQ ID NO: 12 - DNA, 21 bp, Artificial Sequence
  Solyc02g071860-R
atcctgccctatgagagatat

SEQ ID NO: 13 - DNA, 22 bp, Artificial Sequence
  Solyc02g071870-F
atgttgccactaaaaagagctg

SEQ ID NO: 14 - DNA, 23 bp, Artificial Sequence
  Solyc02g071870-R
aggaagattaatacccttaagag

SEQ ID NO: 15 - DNA, 21 bp, Artificial Sequence
  Solyc02g071880-F
atgtatccaaccatagcttgg

SEQ ID NO: 16 - DNA, 25 bp, Artificial Sequence
  Solyc02g071880-R
gggaagctttaccaattcaggtggg

SEQ ID NO: 17 - DNA, 22 bp, Artificial Sequence
  Solyc02g072070-F
tggatcattgtgctccttgagt

SEQ ID NO: 18 - DNA, 20 bp, Artificial Sequence
  Solyc02g072070-R
ccagctcaaggtcagtccag

SEQ ID NO: 19 - DNA, 20 bp, Artificial Sequence
  Solyc02g072250-F
tggctccattgttcctctca

SEQ ID NO: 20 - DNA, 20 bp, Artificial Sequence
  Solyc02g072250-R
tgaacaggagaaacagggca

SEQ ID NO: 21 - DNA, 20 bp, Artificial Sequence
  Solyc02g072310-F
cattgggtggccactactcc

SEQ ID NO: 22 - DNA, 20 bp, Artificial Sequence
  Solyc02g072310-R
ttcagcaggaatcggaccag

SEQ ID NO: 23 - DNA, 20 bp, Artificial Sequence
  Solyc02g072390-F
acaacttggcaacctctcct

SEQ ID NO: 24 - DNA, 21 bp, Artificial Sequence
  Solyc02g072390-R
gttgttgccgagtgcaagatt

SEQ ID NO: 25 - DNA, 21 bp, Artificial Sequence
  Solyc02g072400-F
gcaacttgcttagccatgaat

SEQ ID NO: 26 - DNA, 21 bp, Artificial Sequence
  Solyc02g072400-R
cgcaaacgagaaaactcaggt

SEQ ID NO: 27 - DNA, 21 bp, Artificial Sequence
  Solyc02g072430-F
ctttgttcagactggggagat

SEQ ID NO: 28 - DNA, 22 bp, Artificial Sequence
  Solyc02g072430-R
tcacttctttccaagactccga

SEQ ID NO: 29 - DNA, 20 bp, Artificial Sequence
  Solyc02g072440-F
accacgcagcatatccaact

SEQ ID NO: 30 - DNA, 22 bp, Artificial Sequence
  Solyc02g072440-R
gagctgggtatagatccaccaa

SEQ ID NO: 31 - DNA, 22 bp, Artificial Sequence
  Solyc02g072470-F
ggcaatctgcctcaagaaatgg

SEQ ID NO: 32 - DNA, 21 bp, Artificial Sequence
  Solyc02g072470-R
ggatggaggaacagtaccagt

SEQ ID NO: 33 - DNA, 20 bp, Artificial Sequence
  Solyc02g072480-F
tgtcaagcaactggacctca

SEQ ID NO: 34 - DNA, 20 bp, Artificial Sequence
  Solyc02g072480-R
agtgctggagttctggcaaa

SEQ ID NO: 35 - DNA, 25 bp, Artificial Sequence
  Solyc02g072520-F
acctccacttgaattcatctactct

SEQ ID NO: 36 - DNA, 22 bp, Artificial Sequence
  Solyc02g072520-R
ccatgagactatgccagtgagg

SEQ ID NO: 37 - DNA, 20 bp, Artificial Sequence
  Solyc02g076660-F
gggggcagatggaacactta

SEQ ID NO: 38 - DNA, 22 bp, Artificial Sequence
  Solyc02g076660-R
agcaccaaaaagacggttttca

SEQ ID NO: 39 - DNA, 24 bp, Artificial Sequence
  Solyc12g015870-F
ccatccaaatgctccgatcgatga

SEQ ID NO: 40 - DNA, 24 bp, Artificial Sequence
  Solyc12g015870-R
tgcctctcaatgaagccttgttgc

SEQ ID NO: 41 - DNA, 20 bp, Artificial Sequence
  Solyc02g072470qRT-F
tgatcagtccgcgcttcttt

SEQ ID NO: 42 - DNA, 20 bp, Artificial Sequence
  Solyc02g072470qRT-R
gtgcctagagccacaagtga

SEQ ID NO: 43 - DNA, 24 bp, Artificial Sequence
  SL2.40ch0232439779
cagacccaaatacagttgtagatg

SEQ ID NO: 44 - DNA, 25 bp, Artificial Sequence
  SL2.40ch0232440140
ctagcaaattatatcatgacattcg

SEQ ID NO: 45 - DNA, 24 bp, Artificial Sequence
  SL2.40ch0234555022
gaattggcttactaaagtgtttgg

SEQ ID NO: 46 - DNA, 26 bp, Artificial Sequence
  SL2.40ch0234555443
caagcaaagcaacctcattcaactcc

SEQ ID NO: 47 - DNA, 21 bp, Artificial Sequence
  SL2.40ch0234831393
agccaactttggttgaaggtg

SEQ ID NO: 48 - DNA, 22 bp, Artificial Sequence
  SL2.40ch0234831605
ctccttagggtagagattcgcc

SEQ ID NO: 49 - DNA, 22 bp, Artificial Sequence
  SL2.40ch0235089735
aatgtggcatcgcacgaagcac

SEQ ID NO: 50 - DNA, 23 bp, Artificial Sequence
  SL2.40ch0235090118
cattatccattgggaatttctcc

SEQ ID NO: 51 - DNA, 25 bp, Artificial Sequence
  SL2.40ch0235363629
ctttctattaatctcttctccccca

SEQ ID NO: 52 - DNA, 24 bp, Artificial Sequence
  SL2.40ch0235364150
actatggaatggttagtggaacct

SEQ ID NO: 53 - DNA, 24 bp, Artificial Sequence
  SL2.40ch0235640449
gatttggttgcctgaaagttattc

SEQ ID NO: 54 - DNA, 25 bp, Artificial Sequence
  SL2.40ch0235641543
ctctttgtatataattctttgagat

SEQ ID NO: 55 - DNA, 20 bp, Artificial Sequence
  SL2.40ch0235640674
ggctcaatcccgaagtggtt

SEQ ID NO: 56 - DNA, 25 bp, Artificial Sequence
  SL2.40ch0235641161
tccaagtcttattggacaactttct

SEQ ID NO: 57 - DNA, 25 bp, Artificial Sequence
  SL2.40ch0236160948
gtttttgggtttattatgatcattg

SEQ ID NO: 58 - DNA, 21 bp, Artificial Sequence
  SL2.40ch0236161444
gtgtcaagaacactggttgca

SEQ ID NO: 59 - DNA, 25 bp, Artificial Sequence
  SL2.40ch0236160948
gtttttgggtttattatgatcattg

SEQ ID NO: 60 - DNA, 24 bp, Artificial Sequence
  SL2.40ch0236161948
cgtaggttatgggttgctcgagtc

SEQ ID NO: 61 - DNA, 20 bp, Artificial Sequence
  SL2.40ch0236413739
ttttgagtgcacttgctcct

SEQ ID NO: 62 - DNA, 23 bp, Artificial Sequence
  SL2.40ch0236414522
gtcactagatgctaaagaagggc

SEQ ID NO: 63 - DNA, 20 bp, Artificial Sequence
  SL2.40ch0236413739
ttttgagtgcacttgctcct

SEQ ID NO: 64 - DNA, 21 bp, Artificial Sequence
  SL2.40ch0236414160
actgcgttagttgggagagag

SEQ ID NO: 65 - DNA, 25 bp, Artificial Sequence
  SL2.40ch0236700673
gtgaagtctcgagaatatatttgtc

SEQ ID NO: 66 - DNA, 25 bp, Artificial Sequence
  SL2.40ch0236701226
ggcaagaggaacatgttccgtaagg

SEQ ID NO: 67 - DNA, 22 bp, Artificial Sequence
  SL2.40ch0238827214
tgagttgtcgacgtctatgatg

SEQ ID NO: 68 - DNA, 21 bp, Artificial Sequence
  SL2.40ch0238827889
aggatacttgagcaaaaggct

SEQ ID NO: 69 - DNA, 22 bp, Artificial Sequence
  SL2.40ch0238827214
tgagttgtcgacgtctatgatg

SEQ ID NO: 70 - DNA, 24 bp, Artificial Sequence
  SL2.40ch0238827568
acgtcccaattcccataaattact

SEQ ID NO: 71 - DNA, 22 bp, Artificial Sequence
  SL2.40ch0242479171
ccttttcaattaccctcgctgg

SEQ ID NO: 72 - DNA, 25 bp, Artificial Sequence
  SL2.40ch0242479799
aagctttgtaactccaagtatgttt

SEQ ID NO: 73 - protein, 22 aa, Artificial Sequence
  Flg22 region
QRLSTGSRINSAKDDAAGLQIA

SEQ ID NO: 74 - protein, 60 aa, Artificial Sequence
  Flg22 region
MALTVNTNVASLNVQKNLGRASDALSTSMTRLSSGLKINSAKDDAAGLQIATKITSQIRG

SEQ ID NO: 75 - protein, 60 aa, Artificial Sequence
  FlgII-28 region
QTMAIKNANDGMSLAQTAEGALQESTNILQRMRELAVQSRNDSNSSTDRDALNKEFTAMS

SEQ ID NO: 76 - protein, 60 aa, Artificial Sequence
  FlgII-28 region
QTMAIKNANDGMSLAQTAEGALQESTNILQRMRELAVQFRNDSNSSTDRDALNKEFTAMS

SEQ ID NO: 77 - protein, 60 aa, Artificial Sequence
  FlgII-28 region
QTMAIKNANDGMSLAQTAEGALQESTNILQRMRELAVQSRNDSNSATDREALNKEFTAMS

SEQ ID NO: 78 - protein, 60 aa, Artificial Sequence
  Csp22 region
MSNRQTGTVKWFNDEKGFGFITPQGGGDDLFVHFKAIESDGFKSLKEGQTVSFVAAKGQK

SEQ ID NO: 79 - protein, 60 aa, Artificial Sequence
  Csp22 region
MSNRQTGTVKWFNDEKGFGFITPQSGDDLFVHFKAIQSDGFKSLKEGQQVSFIATRGQKG

SEQ ID NO: 80 - protein, 60 aa, Artificial Sequence
  Csp22 region
MAERQSGTVKWFNDEKGFGFITPESGPDLFVHFRAIQGSGFKSLKEGQKVTFIAVQGQKG

SEQ ID NO: 81 - protein, 60 aa, Artificial Sequence
  Csp22 region
MAERQSGTVKWFNDEKGFGFITPESGPDLFVHFRAIQGNGFKSLKEGQKVTFIAVQGQKG

SEQ ID NO: 82 - protein, 60 aa, Artificial Sequence
  Csp22 region
MGNRDTGTVKWFNTSKGFGFISRDSGDDIFVHFRAIRGEGHRVLVEGQRVEFSVMNRDKG

SEQ ID NO: 83 - protein, 60 aa, Artificial Sequence
  Csp22 region
MLNGKVKWFNNAKGYGFILEDGKPNEDLFAHYSAIQMDGYKTLKAGQSVRFEIIQGPKGL

SEQ ID NO: 84 - protein, 60 aa, Artificial Sequence
  Csp22 region
MLNGKVKWFNNAKGYGFILEDGKPDEDLFAHYSAIQMDGYKTLKAGQPVRFEIIQGPKGL

SEQ ID NO: 85 - protein, 60 aa, Artificial Sequence
  Csp22 region
MTDDQLVDTGFVNSYDSFKGFGFIRREKGRDVFFFYDDVEDVVNGIAMGDVVRFEVHEEP

SEQ ID NO: 86 - protein, 1212 aa, Solanum lycopersicum
MTLMEKSLFYSQLAFLLLQCIVTSLAIKTETNITTDQSALLSLKSHIISDPFQLLSKSWSQDTSVCNWIGVTCGSRHNRVTSLNISNMGITGTIPQLFGNLTFLVSLDLDSNNFFGNLPQEMVRLRRLKLMKLSYNNFSGEVPSWFGFLAQLEVLTLKNNSFTGLIPSSLSNISNLEALDLAFNTLEGNIPKDIGNLKNLRGLNLGHNNLTGTVPPSFSNATKLEKLILSYNFLHGNIPNEMGDLQNLNWLIIENNQLTGSIPFSIFNISTLESIGFSQNGLSGDLPDDLCDHLPILKGLYLSFNKLQGHMPQSLSRCYELQLLSLSNNDFDGPIPSEIGMLSNLQTLYLGFNRFTGEIPQEIGDLVNLVMIGMERNQLTGSIPKSIFNISSLQLLSLQNNNFTGSLSREIGNLTMLQGLYLGQNMLTGEIPKEVSNLIELVDIDLGSNRFSGSFPMGIFNISGLRLIDLTDNTLSGTLPSSIGSMLPNIELLYLGGLTNLAGSMPHSLSNCSRLTALDLSLNKLSGSIPNSLGDLTLLQTLNLMENNLSSDQSSQELNFLTSLTNCRNLKQLSLSFNPLNGMLPPSVGNLSTSLEKILASDCQIKGDIPNDIGNLSSLIYLFLYGNRLTGPIPGTLGSLGRLQEFSLANNRLKGSIGDSLCKMQNLGNIYLGENQFSGLVPNCLGNVTSLRGIKLNSNRLSSNIPLSLGNLKDLLELDLSSNNMSGSLPAEIGNLRVAIRIDLSHNQFSNGIPREIGDMQNLIYLSLAQNKLQGSIPDSIGSIPSLEFLDLSNNNLSGSIPMSLEKLRYLNYFNVSFNSLQGEIPFSGPFKNLSSLSFMFNEALCGAPRFHVPSCPTSSNHRSKRKKLLLIVFPLLGAAVTIVFVTLAFVWMRYRKEGNVPVQADLLATRERISYYEIIQATNDFSESNFIGSGSFGSVYKGILINGTIIAVKVFNLQVEGAFKSFETECEVLRNLRHRNLTKVISSCSNLDFKALVLEYMPNGSLEKWLYSHNYFLDILQRLSIMIDVACALEYLHHGCSAPVIHCDLKPSNVLLDENMVAHLSDFGISKLLSEDESDLHTKTLATFGYIAPEYGREGLLSLKCDVYSYGIMLMETFTRRRPNDEIFDEDLSLKKWVSDSLPEATIKVVDANFLTPEDEKFMEKIDCVASIMKVALDCSAESPEERIYMKDVVGTLQKIKIQLLSCSASA

SEQ ID NO: 87 - protein, 1212 aa, Solanum lycopersicum
MTLMEKSLFYSQLAFLLLQCIVTSLAIKTETNITTDQSALLSLRSHIISDPFQLLSKSWSQDTSVCNWIGVTCGSRHNRVTSLNISNMGITGTIPQLFGNLTFLVSLDLASNNFYGNLPQEMVRLHRLKLMKLSYNNFSGEVPSWFGFLAQLQVLTLKNNSFTGLIPSSLSNISNLEALDLAFNALEGNIPKDIGNLKNLRGLNLGHNNLTGSVPPSFSNATKLEKLILSYNFLHGNIPNEIGDLHNLNWLIIETNQLTGSIPFSIFNISTLESIGFSQNGLSGDLPDDLCDHLPILKGLYLSFNKLQGHMPRSLSRCYELQLLSLSNNDFDGPIHSEIGMLSNLQTLYLGFNRFTGEIPQEIGDLVNLVMIGMERNQLTGSIPKSIFNISSLQLLSLQNNNLTGSLSREIGNLTMLQGLYLGQNMLTGEIPKEVSNLIELVDIDLGSNRFSGSFPMGIFNISGLRLIDLTDNTLSGTLPSSIGSMLPNIELLYLGGLTNLAGSMPLSLSNCSRLTALDLSLNKLSGSIPNSLGDLTLLQTLNLMENNLSSDQSSQELNFLTSLTNCRNLKQLSLSFNPLNGMLPASVGNLSTSLEKILASDCQINGDIPNDIGNLSSLIYLYLYGNRLTGPIPGTLGSLGRLQEFSLANNRLKGSIGDSLCKMQNLGNIYLGENQFSGIVPYCLGNVTSLRGIKLNSNRLSSNIPLSLGNLKDLLELDLSSNNMSGSLPAEIGNLRVAIRIDLSHNQFSNGIPREIGDMQNLIYLSLAQNKLQGSIPDSIGSIPSLEFLDLSNNNLSGSIPMSLEKLRYLNYFNVSFNSLQGEIPFSGPFKNLSSLSFMFNEALCGAPRFHVPSCPTSSNHRSKRKKLLLIVFPLLGAAVTIVFVTLAFVWMRYRKERKIPVQADLLATRERISYYEIIQATNDFSESNFIGSGSFGSVYKGILIDETIIAVKVFNLQVEGAFKSFETECEVLRNLRHRNLTKVISSCSNLDFKALVLEYMPNGSLEKWLYSHNYFLDILQRLSIMIDVACALEYLHHGCSAPVIHCDLKPSNVLLDENMVAHVSDFGISKLLSEDESDLHTKTLATFGYIAPEYGREGLVSLKCDVYSYGIMLMETFTRRRPNDEIFNEDLSLKKWVSDSLPEATIKVVDANLLTPEDEKFMERIDCVASIMKVALDCSAESPEERVNMKDVVGILQKIKIQLLSCYAST

(19) United States
(12) Patent Application Publication
(10) Pub. No.: US 2017/0107531 A1
(43) Pub. Date: Apr. 20, 2017

(54) ISOLATED NUCLEIC ACIDS AND QUANTITATIVE TRAIT LOCI (QTL) FROM S. HABROCHAITES AND METHODS OF USE THEREOF FOR INCREASING RESISTANCE TO BACTERIAL SPECK DISEASE IN TOMATO AND OTHER PLANTS

(71) Applicant: Boyce Thompson Institute, Ithaca, NY (US)

(72) Inventors: Gregory Martin, Ithaca, NY (US); Susan Strickler, Ithaca, NY (US); Zhilong Bao, Gainesville, FL (US)

(21) Appl. No.: 15/254,370

(22) Filed: Sep. 1, 2016

Related U.S. Application Data
(60) Provisional application No. 62/213,409, filed on Sep. 2, 2015.

(51) Int. Cl. C12N 15/82; C12N 9/12

(52) U.S. Cl. CPC C12N 15/8281; C12N 9/12; C12Y 207/00

(57) ABSTRACT

Compositions and methods for increasing disease resistance in plants, particularly tomato plants are disclosed.

### CROSS-REFERENCE TO RELATED APPLICATIONS

The present application claims the benefit of U.S. Provisional Patent Application No. 62/213,409, filed Sep. 2, 2015, the entire disclosure of which is incorporated by reference herein.

### STATEMENT REGARDING FEDERAL SPONSORED RESEARCH OR DEVELOPMENT

This invention was made with government support under Grant Number 10S-1025642 awarded by the National Science Foundation. The government has certain rights in the invention.

### FIELD OF THE INVENTION

This invention relates to the fields of transgenic plants and disease resistance. More specifically, the invention provides compositions and methods of increasing plant resistance to bacterial speck disease.

### BACKGROUND OF THE INVENTION

Several publications and patent documents are cited throughout the specification in order to describe the state of the art to which this invention pertains. Each of these citations is incorporated by reference herein as though set forth in full.

Bacterial speck disease of tomato is caused by Pseudomonas syringae pv. tomato (Pst) and can be a problem in production areas throughout the world where moist, cool conditions occur (Pedley and Martin, 2003, Young, et al., 1986). The disease manifests as necrotic lesions (specks) on all aerial parts of the plant and can result in decreased fruit quality and yield resulting in significant economic losses (Jones, 1991). The experimental tractability of both Pst and tomato has facilitated the study of their interaction and led to many insights into the molecular basis of bacterial pathogenesis and the plant immune system (Martin, 2012, Velasquez and Martin, 2013).

Two races of Pst are defined based upon the ability of the host resistance (R) protein Pto to recognize and confer resistance to them (Jones, 1991, Pedley and Martin, 2003). Race 0 strains translocate the type III effectors AvrPto and/or AvrPtoB into the plant cell where they are recognized by Pto, whereas race 1 strains either lack these effectors, do not accumulate the proteins, or have variants that are not recognized by Pto (Kunkeaw, et al., 2010, Lin, et al., 2006). Genome sequences are available for the widely-studied Pst strain DC3000 (race 0) and five other Pst strains, including two race 1 strains, T1 and NY-T1 (Almeida, et al., 2009, Buell, et al., 2003, Jones, et al., 2015)(http://pseudomonas-syringae.org). Recently, the tomato genome has been sequenced, further enhancing the use of this species for understanding the molecular basis of many phenotypes including plant immunity (Tomato Genome Consortium, 2012).

Plants employ two inter-related immune systems to combat pathogen attack (Dodds and Rathjen, 2010). In the first, the extracellular domains of host transmembrane pattern recognition receptors (PRRs) detect microbe-associated molecular patterns (MAMPs), which are typically conserved molecules required for the microbial lifestyle (Zipfel, 2014). Subsequent signaling events activate pattern-triggered immunity (PTI). Pathogens adapted to a particular host deliver virulence proteins (effectors) into the plant cell where they act in a variety of ways to defeat PTI (Anderson and Frank, 2012, Dou and Zhou, 2012). At this stage, a second immune system can be deployed in which intracellular host receptors (R proteins) recognize, either directly or indirectly, the presence of a pathogen effector to activate effector-triggered immunity (ETI) (Maekawa, et al., 2011). In a further cycle of this molecular ‘arms race’ some pathogens have evolved effectors that interfere with ETI (Guo, et al., 2009, Rosebrock, et al., 2007, Wei, et al., 2015). Numerous details about the mechanisms underlying each of these steps are known and their study constitutes an active area of research (Boller and Felix, 2009).

In tomato, the PRRs FLS2 and FLS3 detect the presence of the Pst flagellin-derived MAMPs Flg22 and FlgII-28, respectively (Cai, et al., 2011, Clarke, et al., 2013, Hind, et al., 201x, Robatzek and Wirthmueller, 2013, Veluchamy, et al., 2014). Tomato also responds to bacterial Csp22, derived from cold shock protein (Felix and Boller, 2003, Veluchamy, et al., 2014)) although its cognate PRR has not yet been reported. FLS2 and FLS3 act with the co-receptor BAK1 to activate PTI, which can be monitored upon MAMP treatment through the generation of reactive oxygen species (ROS), activation of MAP kinases (MAPKs), defense gene expression, and ultimately inhibition of bacterial population growth (Chakravarthy, et al., 2009, Chinchilla, et al., 2007, Heese, et al., 2007, Hind, et al., 201x, Nguyen, et al., 2010). The Pst effectors AvrPto and AvrPtoB interfere with the FLS2/BAK1 and FLS3/BAK1 complexes and thereby impede PTI, allowing progression of bacterial speck disease (Cheng, et al., 2011, Martin, 2012, Shan, et al., 2008, Xiang, et al., 2008).

The resistance protein Pto, a serine/threonine protein kinase, binds to either AvrPto or AvrPtoB and acts with the Prf NB-LRR protein to activate ETI which is associated with transcriptional reprogramming, localized programmed cell death (PCD), and inhibition of pathogen growth, among other responses (Oh and Martin, 2011, Pombo, et al., 2014, Salmeron, et al., 1996, Xing, et al., 2007). In addition to its effectiveness against race 0 strains of Pst, Pto/Prf-mediated ETI can also potentially suppress infection of tomato by diverse P. syringae pathovars that express avrPto or avrPtoB (Lin and Martin, 2007).

Extensive natural variation is observed among tomato germplasm for fruit and leaf morphology, plant architecture, and other traits (Goldman, 2008, Male, 1999). A recent screen of 14 heirloom varieties (also called heritage varieties) for generation of ROS upon treatment with Flg22, FlgII-28 or Csp22 identified natural variation for responses to each of these MAMPs (Veluchamy, et al., 2014). The twelve wild relatives of tomato (in Solanum sect. Lycopersicon) also provide a valuable resource for the identification of genes contributing to plant immunity (Grandillo, et al., 2011, Peralta, et al., 2006). Many of these wild relatives can be readily crossed with cultivated tomato allowing such genes to be introgressed into existing varieties. For example, Pto was identified in Solanum pimpinellifolium and has been introduced into many processing tomatoes (Pedley and Martin, 2003). At least three other wild tomato species are also known to have the Pto gene (Riely and Martin, 2001, Rose, et al., 2005).

The Pto gene has been reported to be present in 14 of the top 20 processing varieties in California and to be used on more than 60% of the state acreage to protect the crop from bacterial speck disease (Pedley and Martin, 2003). The gene has been remarkably effective for over 20 years possibly because Pst strains that lack AvrPto or AvrPtoB are less virulent (Lin and Martin, 2005). Nevertheless, race 1 strains now predominate in many tomato-growing regions and there have been an increasing number of reports of the breakdown of Pto-mediated resistance (Arredondo and Davis, 2000, Buonaurio, et al., 1996, Cai, et al., 2011, Kunkeaw, et al., 2010). It would therefore be useful to identify and characterize sources of resistance to race 1 strains to both combat the disease and possibly lead to novel insights into the plant immune system.

### SUMMARY OF THE INVENTION

In accordance with the present invention, a method for producing a plant exhibiting increased pathogen resistance is provided. An exemplary method entails introducing a qRph1 nucleic acid into a recipient plant cell, the nucleic acid encoding at least one protein useful for conferring resistance to at least one plant pathogen, wherein the cell exhibits increased pathogen resistance when compared to wild type plant cells lacking the qRph1 nucleic acid. In a preferred embodiment, the method results in plants which exhibit increased resistance to Pseudomonas syringae pv tomato (Pst), and the cell is a tomato plant cell. In a particularly preferred embodiment, the at least one protein is a receptor like protein kinase (RLK) encoded by Soly02g072470.

In another aspect of the invention, a vector comprising an RLK encoded by Solyc02g072470 is provided. The invention also includes transgenic host cells and plants comprising the RLK. Surprising, such plants exhibit increased resistance to Pst relative to plants lacking said vector.

### BRIEF DESCRIPTION OF THE DRAWINGS

FIG. 1A-FIG. 1B. Solanum habrochaites accession LA2109 is resistant to P. syringae pv. tomato strain T1 and was collected in a region of southern Ecuador that has many T1-resistant S. habrochaites accessions. (FIG. 1A) The map and close-up show the collection sites of 36 accessions of S. habrochaites (also see Table 1 and Table 2). Triangle, rectangle and red circle symbols indicate accessions that were susceptible, moderately resistant or resistant to T1, respectively. The photograph and inset show LA2109 four days after inoculation with T1 (no symptoms of disease were observed; see FIG. 1B for inoculation of LA2109 with NY-T1). P. syringae pv. tomato strain NY-T1 is highly virulent on LA2109. (FIG. 1B) Bacterial speck disease on LA2109 four days after inoculation with NY-T1. Inset shows close-up of the disease symptoms.

FIG. 2A-FIG. 2D. Solanum habrochaites accession LA2109 has both Pto-dependent and independent resistance to P. s. pv. tomato. Bacterial speck disease on LA2109 four days after inoculation with DC3000 (FIG. 2A) or DC3000ΔavrPtoΔavrPtoB (FIG. 2B). Insets show close up of a representative leaflet. (FIG. 2C) Bacterial populations in leaves of LA2109 after inoculation with T1 or DC3000ΔavrPtoΔavrPtoB. Error bars indicate standard deviation. The asterisk indicates a statistically significant difference as determined by a Student's t test (P<0.05). Comparison of peptide MAMPs in T1, NY-T1 and DC3000 (FIG. 2D). Flg22: SEQ ID NO: 73; Flagellin-Pto_T1, Flagellin-Pto_NY-T1, Flagellin-Pto_DC3000: SEQ ID NO: 74; FlgII-28: SEQ ID NO: 1; Flagellin-Pto_T1: SEQ ID NO: 75; Flagellin-Pto_NY-T1: SEQ ID NO: 76; Flagellin-Pto_DC3000: SEQ ID NO: 77; Csp22: SEQ ID NO: 2; PSPTO_2376-CapA-DC3000, NYT1-5113480_5113692, PtoT1-EEB58236.1-capA: SEQ ID NO: 78; PSPTO_4145-DC3000, NYT1-1212985_1213194, PSPTOT1_5716-capB: SEQ ID NO: 79; PtoT1-EEB60570.1, NYT1-3206290_3206502: SEQ ID NO: 80; PSPTO_1274-DC3000: SEQ ID NO: 81; PSPTO_3984-DC3000, NYT1-1802441_1803049, PtoT1-EEB59732.1: SEQ ID NO: 82; PtoT1-EEB58713.1, NYT1-2726888_2727157: SEQ ID NO: 83; PSPTO_3355-DC3000′ SEQ ID NO: 84; PSPTO_3929-DC3000, NYT1-, PtoT1-EEB59053.1: SEQ ID NO: 85.

FIG. 3A-FIG. 3E. The resistance of LA2109 to P. s. pv. tomato Pst19 strain does not involve AvrPtoB or Pto. (FIG. 3A) Bacterial speck disease on leaves of LA2109 four days after inoculation with Pst19 or Pst19ΔavrPtoB. (FIG. 3B) Bacterial populations of Pst19 and Pst19ΔavrPtoB in leaves of LA2109. Error bars indicate standard deviation. Testing the possible response of LA2109 to FlgII-28, effector HopAO1, and coronatine. (FIG. 3C) FlgII-28 from both T1 and DC3000 elicits the same level of reactive oxygen species (ROS) in LA2109 whereas the FlgII-28 from NY-T1 is inactive. ROS was measured as relative light units using a luminescence-based assay. (FIG. 3D) The addition of HopAO1 to T1 does not alter the resistance of LA2109. Strain NY-T1 was included as a virulent control. All strains were inoculated at 2×10⁵ CFU/mL. (FIG. 3E) Coronatine (1.25 nM) was syringe-infiltrated into leaves of LA2109 and Moneymaker (red arrows). Leaves of LA2109 were not observably more sensitive to this phytotoxin.

FIG. 4A-FIG. 4B. Resistance in RG-PtoS x LA2109 F2 plants was categorized into three phenotypes. (FIG. 4A) Symptoms of bacterial speck disease on leaves of F2 plants representative of the three phenotypes observed. (FIG. 4B) The disease categories and number of F2 plants in each category that were subjected to Illumina sequencing are shown.

FIG. 4C-FIG. 4D. Two strains of P. syringae pv. apii (a pathogen of celery—FIG. 4C) and two from P. syringae pv. persicae (a pathogen of peach—FIG. 4D) that met these criteria were inoculated onto LA 2109. Each of these strains caused numerous necrotic lesions on RG-PtoR and little or no symptoms of disease on LA2109. Thus, LA2109 might recognize a feature present in a diverse range of P. syringae pathovars.

FIG. 5A-FIG. 5C. QTL associated with T1 resistance detected by low depth whole-genome sequencing of F2 individual plants and linkage analysis. (FIG. 5A) Scheme to identify QTL involved in T1 resistance from low coverage data. (FIG. 5B) Log-likelihood plot showing the 12 chromosomes of tomato. The resistance QTL on chromosome 2 is located between 35,128,470 bp and 40,887,114 bp (a 5.8 Mb region) and the QTL on chromosome 8 is located between 2,089,886 bp and 54,513,866 bp (a 52.4 Mb region). (FIG. 5C) Both RG-PtoS and LA2109 have sequences corresponding to Solyc02g072470 and Solyc02g072480 in their genomes. DNA sequences of Solyc02g072470 and Solyc02g072480 were able to be PCR-amplified from genomic DNA of RG-PtoS and LA2109 with the same primers as were used in FIG. 7A.

FIG. 6A-FIG. 6D. Fine mapping of the candidate QTL qRph1 on chromosome 2. (FIG. 6A) Confirmation using 49 resistant F2 plants that a region on chromosome 2 is linked to T1 resistance. (FIG. 6B) Linkage analysis identified the QTL boundaries on chromosome 2 using low coverage whole genome sequence data. (FIG. 6C) Fine-mapping with 85 resistant plants isolated from 423 F2 plants was used to delimit the candidate region (between the dashed lines) to between chromosome 2 coordinates 35,640,449 bp and 36,700,673 bp (1,060 kb) in the reference tomato genome Heinz SL2.40. A total of 17 RLK, 3 RLP and no NB-LRR genes are annotated in this region. Triangles and boxes indicate positions of these RLKs and RLPs, respectively. The arrow points to the candidate for qRph1 (Solyc02g072470). The locations of SlFLS2.1 and SlFLS2.2 are also shown. (FIG. 6D) Transcript abundance of Solyc02g072470 in S. habrochaites accessions collected in the same geographical region as LA2109. Transcript abundance of Solyc02g072470 was determined by RT-PCR in S. habrochaites accessions that are resistant to T1 (see FIG. 1) and collected from regions close to the LA2109 collection site. Leaf tissues used for RNA extractions were harvested from individual plants that were confirmed to be resistant to T1 (FIG. 1). PCR reactions used 35 cycles for Solyc02g072470 and 25 cycles for a control gene Solyc12g015870.

FIG. 7A-FIG. 7D. Transcript abundance of RLK- and RLP-encoding genes in the candidate region of chromosome 2. The transcript abundance in leaves of the genes encoding RLKs or RLPs in the 1,060 kb candidate region was determined by RT-PCR in RG-PtoR (R), RG-PtoS (S) and LA2109 (LA). Each RT-PCR was for 35 cycles except the control gene Solyc12g015870 (Calcineurin B-like protein) which was for 25 cycles. The asterisk indicates a possible alternatively-spliced transcript of Solyc02g072440. See Tables S4 and 5 for additional details of the genes. Details of Solyco2g072470. (FIG. 7B) Gene structure of Solyc02g072470. Black boxes indicate exons, and lines indicate introns. (FIG. 7C) Features of the Solyc02g072470 protein. Leucine-rich repeats (LRR) and kinase domains as predicted by PROSITE. (FIG. 7D) Alignment of the predicted amino acid sequences of Solyc02g072470 in Heinz 1706 (SEQ ID NO: 86) and LA2109 (SEQ ID NO: 87).

FIG. 8A-FIG. 8D. Transcript abundance of Solyc02g072470 in leaves treated with FlgII-28 or Csp22. (FIG. 8A) Solyc02g072470 transcript abundance 6 hours after treatment of leaves with 1 μM FlgII-28 or 10 mM MgCl₂ (mock) was analyzed by qRT-PCR. (FIG. 8B) Solyc02g072470 transcript abundance 6 hours after treatment of leaves with 10 μM Csp22 or 10 mM MgCl₂ (mock) was analyzed by qRT-PCR. SICBL1 (Calcineurin B-like protein, Solyc12g015870) transcript abundance in RG-PtoS after mock treatment in was used for all comparisons to calculate the relative expression. Shown are data from one of two experiments both of which gave similar results. Error bars indicate the standard deviation. The letters a, b and c indicate statistically significant differences compared with RG-PtoS after the mock treatment as determined by a Student's t test (P<0.05). (FIG. 8C) Phylogenetic analysis of Solyc02g072470 and similar genes in tomato. 17 RLK genes were chosen based on the similarity of their nucleotide sequences to Solyc02g072470. All genes were identified based on a BLAST analysis of the tomato genome using Solyc02g072470 nucleotide sequence. (FIG. 8D) 21 RLKs were likewise chosen based on the similarity of their amino acid sequences to Solyc02g072470. Solyc02g070890 (SIFLS2.1) and Solyc02g070910 (SIFLS2.2) were included in both trees as outgroups.

### DETAILED DESCRIPTION OF THE INVENTION

Bacterial speck disease caused by Pseudomonas syringae pv. tomato (Pst) is a persistent problem on tomato. Resistance against race 0 Pst strains is conferred by the Pto protein which recognizes either of two pathogen effectors, AvrPto or AvrPtoB. However, current tomato varieties do not have resistance to the increasingly common race 1 strains which lack these effectors. We identified accessions of Solanum habrochaites that are resistant to the race 1 strain T1. Genome sequence comparisons of T1 and two Pst strains that are virulent on these accessions suggested that known microbe-associated molecular patterns (MAMPs) or effectors are not involved in the resistance. We developed an F2 population from a cross between one T1-resistant accession, LA2109, and a susceptible tomato cultivar to investigate the genetic basis of this resistance. Linkage analysis using whole genome sequence of 58 F2 plants identified quantitative trait loci, qRph1, in a 5.8 Mbp region on chromosome 2, and qRph2, in a 52.4 Mbp region on chromosome 8 which account for 24% and 26% of the phenotypic variability, respectively. High-resolution mapping of qRph1 confirmed it contributed to T1 resistance and delimited it to a 1,060 kbp region containing 139 genes, including three encoding receptor-like proteins and 17 encoding receptor-like protein kinases (RLKs). One RLK gene, Solyc02g072470, is a promising candidate for qRph1 as it is highly expressed in LA2109 and induced upon treatment with MAMPs. Accordingly, qRph1 is useful for enhancing resistance to race 1 strains.

### DEFINITIONS

The following definitions are provided to facilitate an understanding of the present invention:

“qRph1” refers to quantitative trait loci associated with resistance to bacterial speck disease caused by Pseudomonas syringae pv. tomato (Pst). A receptor like kinase (RLK) encoded by Solyc02g072470 is present in the qRph1 region and is highly expressed in LA2109, which is highly resistant to Pst.

The term “qRph1 function” is used herein to refer to any qRph1 activity, including without limitation expression levels of qRph1, enzymatic activity, and enhancement of disease resistance.

An qRph1 homolog is any protein or DNA encoding the same which has similar structural properties (such as sequence identity and folding) to qRph1. An qRph1 ortholog is any protein or DNA encoding the same which has similar structural properties, and similar function (such as expression, enzymatic activity, and binding) to qRph1.

The term “pathogen-inoculated” refers to the inoculation of a plant with a pathogen.

The term “disease defense response” refers to a change in metabolism, biosynthetic activity or gene expression that enhances a plant's ability to suppress the replication and spread of a microbial pathogen (i.e., to resist the microbial pathogen). Examples of plant disease defense responses include, but are not limited to, production of low molecular weight compounds with antimicrobial activity (referred to as phytoalexins) and induction of expression of defense (or defense-related) genes, whose products include, for example, peroxidases, cell wall proteins, proteinase inhibitors, hydrolytic enzymes, pathogenesis-related (PR) proteins and phytoalexin biosynthetic enzymes, such as phenylalanine ammonia lyase and chalcone synthase. Such defense responses appear to be induced in plants by several signal transduction pathways involving secondary defense signaling molecules produced in plants. Agents that induce disease defense responses in plants include, but are not limited to: (1) microbial pathogens, such as fungi, oomycetes, bacteria and viruses; (2) microbial components and other defense response elicitors, such as proteins and protein fragments, small peptides, β-glucans, elicitins, harpins and oligosaccharides; and (3) secondary defense signaling molecules produced by the plant, such as SA, H₂O₂, ethylene, jasmonates, and nitric oxide.

The terms “defense-related genes” and “defense-related proteins” refer to genes or their encoded proteins whose expression or synthesis is associated with or induced after infection with a pathogen to which the plant is usually resistant.

A “transgenic plant” refers to a plant whose genome has been altered by the introduction of at least one heterologous nucleic acid molecule.

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism.

When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An “isolated nucleic acid” (either DNA or RNA) may further represent a molecule produced directly by biological or synthetic means and separated from other components present during its production.

The terms “percent similarity”, “percent identity” and “percent homology” when referring to a particular sequence are used as set forth in the University of Wisconsin GCG software program.

The term “substantially pure” refers to a preparation comprising at least 50 60% by weight of a given material (e.g., nucleic acid, oligonucleotide, protein, etc.). More preferably, the preparation comprises at least 75% by weight, and most preferably 90 95% by weight of the given compound. Purity is measured by methods appropriate for the given compound (e.g. chromatographic methods, agarose or polyacrylamide gel electrophoresis, HPLC analysis, and the like).

A “replicon” is any genetic element, for example, a plasmid, cosmid, bacmid, phage or virus, that is capable of replication largely under its own control. A replicon may be either RNA or DNA and may be single or double stranded.

A “vector” is any vehicle to which another genetic sequence or element (either DNA or RNA) may be attached so as to bring about the replication of the attached sequence or element.

An “expression operon” refers to a nucleic acid segment that may possess transcriptional and translational control sequences, such as promoters, enhancers, translational start signals (e.g., ATG or AUG codons), polyadenylation signals, terminators, and the like, and which facilitate the expression of a polypeptide coding sequence in a host cell or organism.

The term “oligonucleotide,” as used herein refers to sequences, primers and probes of the present invention, and is defined as a nucleic acid molecule comprised of two or more ribo- or deoxyribonucleotides, preferably more than three. The exact size of the oligonucleotide will depend on various factors and on the particular application and use of the oligonucleotide.

The phrase “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides. The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically. The term “primer” as used herein refers to an oligonucleotide, either RNA or DNA, either single-stranded or double-stranded, either derived from a biological system, generated by restriction enzyme digestion, or produced synthetically which, when placed in the proper environment, is able to functionally act as an initiator of template-dependent nucleic acid synthesis. When presented with an appropriate nucleic acid template, suitable nucleoside triphosphate precursors of nucleic acids, a polymerase enzyme, suitable cofactors and conditions such as appropriate temperature and pH, the primer may be extended at its 3′ terminus by the addition of nucleotides by the action of a polymerase or similar activity to yield a primer extension product. The primer may vary in length depending on the particular conditions and requirement of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

Polymerase chain reaction (PCR) has been described in U.S. Pat. Nos. 4,683,195, 4,800,195, and 4,965,188, the entire disclosures of which are incorporated by reference herein.

The term “promoter region” refers to the 5′ regulatory regions of a gene (e.g., CaMV 35S promoters and/or tetracycline repressor/operator gene promoters).

As used herein, the terms “reporter,” “reporter system”, “reporter gene,” or “reporter gene product” shall mean an operative genetic system in which a nucleic acid comprises a gene that encodes a product that when expressed produces a reporter signal that is a readily measurable, e.g., by biological assay, immunoassay, radio immunoassay, or by calorimetric, fluorogenic, chemiluminescent or other methods. The nucleic acid may be either RNA or DNA, linear or circular, single or double stranded, antisense or sense polarity, and is operatively linked to the necessary control elements for the expression of the reporter gene product. The required control elements will vary according to the nature of the reporter system and whether the reporter gene is in the form of DNA or RNA, but may include, but not be limited to, such elements as promoters, enhancers, translational control sequences, poly A addition signals, transcriptional termination signals and the like.

The terms “transform”, “transfect”, “transduce”, shall refer to any method or means by which a nucleic acid is introduced into a cell or host organism and may be used interchangeably to convey the same meaning. Such methods include, but are not limited to, transfection, electroporation, microinjection, PEG-fusion and the like.

The introduced nucleic acid may or may not be integrated (covalently linked) into nucleic acid of the recipient cell or organism. In bacterial, yeast, plant and mammalian cells, for example, the introduced nucleic acid may be maintained as an episomal element or independent replicon such as a plasmid. Alternatively, the introduced nucleic acid may become integrated into the nucleic acid of the recipient cell or organism and be stably maintained in that cell or organism and further passed on or inherited to progeny cells or organisms of the recipient cell or organism. Finally, the introduced nucleic acid may exist in the recipient cell or host organism only transiently.

The term “selectable marker gene” refers to a gene that when expressed confers a selectable phenotype, such as antibiotic resistance, on a transformed cell or plant.

The term “operably linked” means that the regulatory sequences necessary for expression of the coding sequence are placed in the DNA molecule in the appropriate positions relative to the coding sequence so as to effect expression of the coding sequence. This same definition is sometimes applied to the arrangement of transcription units and other transcription control elements (e.g. enhancers) in an expression vector.

The term “DNA construct” refers to a genetic sequence used to transform plants and generate progeny transgenic plants. These constructs may be administered to plants in a viral or plasmid vector. Other methods of delivery such as Agrobacterium T-DNA mediated transformation and transformation using the biolistic process are also contemplated to be within the scope of the present invention. The transforming DNA may be prepared according to standard protocols such as those set forth in “Current Protocols in Molecular Biology”, eds. Frederick M. Ausubel et al., John Wiley & Sons, 1995.

The term “co-suppression” refers to a process whereby expression of a gene, which has been transformed into a cell or plant (transgene), causes silencing of the expression of endogenous genes that share sequence identity with the transgene. Silencing of the transgene also occurs.

Amino acid residues described herein are preferred to be in the “L” isomeric form. However, residues in the “D” isomeric form may be substituted for any L-amino acid residue, provided the desired properties of the polypeptide are retained. All amino-acid residue sequences represented herein conform to the conventional left-to-right amino-terminus to carboxy-terminus orientation.

The term “isolated protein” or “isolated and purified protein” is sometimes used herein. This term refers primarily to a protein produced by expression of an isolated nucleic acid molecule of the invention. Alternatively, this term may refer to a protein that has been sufficiently separated from other proteins with which it would naturally be associated, so as to exist in “substantially pure” form. “Isolated” is not meant to exclude artificial or synthetic mixtures with other compounds or materials, or the presence of impurities that do not interfere with the fundamental activity, and that may be present, for example, due to incomplete purification, or the addition of stabilizers.

The present invention also includes active portions, fragments, derivatives and functional or non-functional mimetics of qRph1-related polypeptides, or proteins of the invention. An “active portion” of such a polypeptide means a peptide that is less than the full length polypeptide, but which retains measurable biological activity.

A “fragment” or “portion” of an qRph1-related polypeptide means a stretch of amino acid residues of at least about five to seven contiguous amino acids, often at least about seven to nine contiguous amino acids, typically at least about nine to thirteen contiguous amino acids and, most preferably, at least about twenty to thirty or more contiguous amino acids. Fragments of the qRph1-related polypeptide sequence, antigenic determinants, or epitopes are useful for eliciting immune responses to a portion of the qRph1-related protein amino acid sequence for the effective production of immunospecific anti-qRph1 antibodies.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the basic and novel characteristics of the sequence.

The term “tag,” “tag sequence” or “protein tag” refers to a chemical moiety, either a nucleotide, oligonucleotide, polynucleotide or an amino acid, peptide or protein or other chemical, that when added to another sequence, provides additional utility or confers useful properties, particularly in the detection or isolation, of that sequence. Thus, for example, a homopolymer nucleic acid sequence or a nucleic acid sequence complementary to a capture oligonucleotide may be added to a primer or probe sequence to facilitate the subsequent isolation of an extension product or hybridized product. In the case of protein tags, histidine residues (e.g., 4 to 8 consecutive histidine residues) may be added to either the amino- or carboxy-terminus of a protein to facilitate protein isolation by chelating metal chromatography. Alternatively, amino acid sequences, peptides, proteins or fusion partners representing epitopes or binding determinants reactive with specific antibody molecules or other molecules (e.g., flag epitope, c-myc epitope, transmembrane epitope of the influenza A virus hemaglutinin protein, protein A, cellulose binding domain, calmodulin binding protein, maltose binding protein, chitin binding domain, glutathione S-transferase, and the like) may be added to proteins to facilitate protein isolation by procedures such as affinity or immunoaffinity chromatography. Chemical tag moieties include such molecules as biotin, which may be added to either nucleic acids or proteins and facilitates isolation or detection by interaction with avidin reagents, and the like. Numerous other tag moieties are known to, and can be envisioned by the trained artisan, and are contemplated to be within the scope of this definition.

A “clone” or “clonal cell population” is a population of cells derived from a single cell or common ancestor by mitosis.

A “cell line” is a clone of a primary cell or cell population that is capable of stable growth in vitro for many generations.

The following description sets forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”) are used.

II. Preparation of qRph1 Encoding Nucleic Acid Molecules:

Nucleic acid molecules of the invention encoding qRph1 polypeptides may be prepared by two general methods: (1) synthesis from appropriate nucleotide triphosphates, or (2) isolation from biological sources. Both methods utilize protocols well known in the art. The availability of nucleotide sequence information, such as the DNA sequences encoding the proteins present in qRph1, enables preparation of an isolated nucleic acid molecule of the invention by oligonucleotide synthesis. Synthetic oligonucleotides may be prepared by the phosphoramidite method employed in the Applied Biosystems 38A DNA Synthesizer or similar devices. The resultant construct may be used directly or purified according to methods known in the art, such as high performance liquid chromatography (HPLC).

Specific probes for identifying such sequences as the qRph1 encoding sequence may be between 15 and 40 nucleotides in length. For probes longer than those described above, the additional contiguous nucleotides are provided within the reference sequences disclosed herein.

Additionally, cDNA or genomic clones having homology with qRph1 may be isolated from other species using oligonucleotide probes corresponding to predetermined sequences within the qRph1 nucleic acids of the invention. Such homologous sequences encoding qRph1 may be identified by using hybridization and washing conditions of appropriate stringency. For example, hybridizations may be performed, according to the method of Sambrook et al., Molecular Cloning, Cold Spring Harbor Laboratory (1989), using a hybridization solution comprising: 5×SSC, 5×Denhardt's reagent, 1.0% SDS, 100 m/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes 1 hour at 37° C. in 1×SSC and 1% SDS; (4) 2 hours at 42-65° C. in 1×SSC and 1% SDS, changing the solution every 30 minutes.

One common formula for calculating the stringency conditions required to achieve hybridization between nucleic acid molecules of a specified sequence homology (Sambrook et al., 1989) is as follows:

Tₘ=81.5° C.+16.6 Log [Na+]+0.41(% G+C)−0.63(% formamide)−600/#bp in duplex

As an illustration of the above formula, using [Na+], [0.368] and 50% formamide, with GC content of 42% and an average probe size of 200 bases, the T., is 57° C. The T., of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated T., of the hybrid. Wash conditions should be as stringent as possible for the degree of identity of the probe for the target. In general, wash conditions are selected to be approximately 12-20° C. below the T., of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and washed in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and washed in 1×SSC and 0.5% SDS at 65° C. for 15 minutes. A very high stringency hybridization is defined as hybridization in 6×SSC, 5×Denhardt's solution, 0.5% SDS and 100 μg/ml denatured salmon sperm DNA at 42° C., and washed in 0.1×SSC and 0.5% SDS at 65° C. for 15 minutes.

The nucleic acid molecules described herein include cDNA, genomic DNA, RNA, and fragments thereof which may be single- or double-stranded. Thus, nucleic acids are provided having sequences capable of hybridizing with at least one sequence of a nucleic acid sequence, such as selected segments of the sequences encoding qRph1. Also contemplated in the scope of the present invention are methods of use for oligonucleotide probes which specifically hybridize with the DNA from the sequences encoding qRph1 under high stringency conditions. Primers capable of specifically amplifying the sequences encoding qRph1 are also provided. As mentioned previously, such oligonucleotides are useful as primers for detecting, isolating and amplifying sequences encoding the proteins present on qRph1.

Also provided in accordance with the present invention are transgenic plants containing the aforementioned qRph1, or fragments or derivatives thereof. Such transgenic plants exhibiting enhanced disease resistance are described in greater detail below.

III. Preparation of qRph1 Proteins and Antibodies:

The proteins present on qRph1 of the present invention may be prepared in a variety of ways, according to known methods. The protein may be purified from appropriate sources, e.g., plant cells or tissues as described in detail in Example 1. Example 1 describes the isolation of qRph1 from LA2109 and expression of this region in F2 plants.

Once nucleic acid molecules encoding the proteins on qRph1 have been obtained, the qRph1 protein (e.g., RLK, Solyc02g072470) can be produced using in vitro expression methods known in the art. For example, a cDNA or gene may be cloned into an appropriate in vitro transcription vector, such a pSP64 or pSP65 vector for in vitro transcription, followed by cell-free translation in a suitable cell-free translation system, such as wheat germ or rabbit reticulocytes. In vitro transcription and translation systems are commercially available, e.g., from Promega Biotech, Madison, Wis. or BRL, Rockville, Md.

According to a preferred embodiment, larger quantities of the RLK on qRph1 may be produced by expression in a suitable procaryotic or eucaryotic system. For example, part or all of a DNA molecule may be inserted into a plasmid vector adapted for expression in a bacterial cell (such as E. coli) or a yeast cell (such as Saccharomyces cerevisiae), or into a baculovirus vector for expression in an insect cell. Such vectors comprise the regulatory elements necessary for expression of the DNA in the host cell, positioned in such a manner as to permit expression of the DNA in the host cell. Such regulatory elements required for expression include promoter sequences, translation control sequences and, optionally, enhancer sequences.

The qRph1 associated RLK protein produced by gene expression in a recombinant procaryotic or eucaryotic system may be purified according to methods known in the art. In a preferred embodiment, the recombinant protein contains several (e.g., 6 8) histidine residues on the amino or carboxyl termini, which allows the protein to be affinity purified on a nickel column. If histidine tag-vectors are not used, an alternative approach involves purifying the recombinant protein by affinity separation, such as by immunological interaction with antibodies that bind specifically to the recombinant protein. Such methods are commonly used by skilled practitioners.

qRph1 proteins, prepared by the aforementioned methods, may be analyzed according to standard procedures. Methods for analyzing the physical characteristics and biological activity of qRph1 are set forth in U.S. Pat. No. 6,136,552, the disclosure of which is incorporated by reference herein.

The present invention also provides antibodies capable of immunospecifically binding to proteins of the invention. Polyclonal or monoclonal antibodies directed toward the proteins encoded by the qRph1 region may be prepared according to standard methods. Monoclonal antibodies may be prepared according to general methods of Kohler and Milstein, following standard protocols. In a preferred embodiment, antibodies are prepared, which react immunospecifically with various epitopes of such proteins.

IV. Uses of qRph1 Nucleic Acid Molecules and Proteins:

A. Nucleic Acids Encoding qRph1-Related Proteins

Nucleic acids encoding qRph1 proteins may be used for a variety of purposes in accordance with the present invention. DNA, RNA, or fragments thereof encoding qRph1 proteins may be used as probes to detect the presence of and/or expression of such genes. Methods in which nucleic acids encoding qRph1 proteins may be utilized as probes for such assays include, but are not limited to: (1) in situ hybridization; (2) Southern hybridization (3) Northern hybridization; and (4) assorted amplification reactions such as polymerase chain reactions (PCR).

The nucleic acids of the invention may also be utilized as probes to identify related genes from other plant species, animals and microbes. As is well known in the art, hybridization stringencies may be adjusted to allow hybridization of nucleic acid probes with complementary sequences of varying degrees of homology. Thus, nucleic acids encoding qRph1 proteins may be used to advantage to identify and characterize other genes of varying degrees of relation to the genes of the invention thereby enabling further characterization of the molecular mechanisms disease resistance in tomato. Additionally, the nucleic acids of the invention may be used to identify genes encoding proteins that interact with qRph1 proteins (e.g., by the “interaction trap” technique), which should further accelerate identification of the molecular components involved in the disease defense response. Nucleic acid molecules, or fragments thereof, encoding qRph1 genes, for example, may also be utilized to control the production of qRph1 proteins, thereby regulating the amount of protein available to participate in the induction or maintenance of disease resistance in plants. As mentioned above, antisense oligonucleotides corresponding to essential processing sites in qRph1-related mRNA molecules or other gene silencing approaches may be utilized to inhibit qRph1 protein production in targeted cells. Yet another approach entails the use of double-stranded RNA mediated gene silencing. Alterations in the physiological amount of qRph1 proteins may dramatically affect the activity of other protein factors involved in the induction or maintenance of disease resistance.

The nucleic acid molecules of the invention may also be used to advantage to identify mutations in qRph1 encoding nucleic acids from plant samples. Nucleic acids may be isolated from plant samples and contacted with the sequences of the invention under conditions where hybridization occurs between sequences of sufficient complementarity. Such duplexes may then be assessed for the presence of mismatched DNA. Mismatches may be due to the presence of a point mutation, insertion or deletion of nucleotide molecules. Detection of such mismatches may be performed using methods well known to those of skill in the art.

Nucleic acids encoding the qRph1 proteins of the invention may also be introduced into host cells. In a preferred embodiment, plant cells are provided which comprise an qRph1 protein(s) encoding nucleic acid such as RLK on Solyc02g072470 or a variant thereof. Host cells contemplated for use include, but are not limited to, tomato, maize, wheat, rice, potato, barley, canola, bacteria, yeast, and other plant cells. The nucleic acids may be operably linked to appropriate regulatory expression elements suitable for the particular host cell to be utilized. Methods for introducing nucleic acids into host cells are well known in the art. Such methods include, but are not limited to, transfection, transformation, calcium phosphate precipitation, electroporation, lipofection and biolistic methods.

The host cells described above or extracts prepared from them containing qRph1 may be used as screening tools to identify compounds which modulate qRph1 protein function. Modulation of qRph1 activity, for example, may be assessed by measuring alterations in qRph1-disease resistance activity, qRph1 enzymatic activities, or qRph1 expression levels in the presence and absence of a test compound. Test compounds may also be assessed for the induction and/or suppression of expression of genes regulated by qRph1 proteins.

The availability of qRph1 protein encoding nucleic acids enables the production of plant species carrying part or all of an qRph1-related gene or mutated sequences thereof, in single or amplified copies. Transgenic plants comprising any one of the qRph1-related sequences described herein are contemplated for use in the present invention. Such plants provide an in vivo model for examining resistance to Pst and may be particularly useful in elucidating the molecular mechanisms that modulate Pst defense responses.

In another embodiment of the invention, transgenic plants are provided that have enhanced disease resistance. Such transgenic plants may have altered qRph1 activity due to the overexpression or underexpression of qRph1-related genes.

As described above, the qRph1-related protein-encoding nucleic acids are also used to advantage to produce large quantities of substantially pure proteins, or selected portions thereof.

The following materials and methods are provided to facilitate the practice of the present invention.

Wild tomato accessions were obtained from the Tomato Genetics Resource Center (tgrc.ucdavis.edu). The wild tomato accessions, F1, and F2 plants generated from a cross between LA2109 and Rio Grande (RG)-PtoS were grown in Sunshine® MVP soil mix (Sun Gro® Horticulture, Agawam, Mass.). RG-PtoS and RG-PtoR plants were grown in Cornell Plus mix, consisting of 5.7 cu ft peat moss, 12 cu ft vermiculite, 5 lbs lime, 5 lbs Osmocote Plus 15-9-12 and 1 lb 3 oz Uni-Mix 11-5-11 (Everris, Israeli Chemicals Ltd). Plants were grown in the greenhouse and moved to a growth chamber with 25° C., 75% humidity, and 16-hour light after pathogen inoculation.

### MAMP Treatments, Pathogen Inoculation, and Bacterial Growth Assays

Two lateral leaflets on the second leaf of 3-week old tomato plants were syringe-infiltrated with 1 uM FlgII-28 (ESTNILQRMRELAVQSRNDSNSATDREA) (SEQ ID NO. 1) (Cal, et al., 2011) or 10 uM Csp22 (AVGTVKWFNAEKGFGFITPDDG) (SEQ ID NO. 2) (Felix and Boller, 2003) and harvested 6 hrs later. Tissue samples were immediately frozen in liquid nitrogen and kept at −80° C. until use. Six-week-old plants were inoculated with 10⁵ colony-forming units (CFU)/mL P. syringae pv. tomato T1 by vacuum infiltration for the visual monitoring of disease symptoms and 10⁴ CFU/mL for the measurement of bacterial populations. For each replicate, three leaf discs were collected and combined; each experiment involved replicates from three independent plants.

### Preparation of DNA Libraries and Illumina Sequencing

Genomic DNA was extracted using a DNeasy Plant Mini kit (Qiagen) and used to make individual libraries as described previously (Zhong et al., 2011). Briefly, 700 ng of DNA from each plant was fragmented using NEBNext dsDNA Fragmentase (New England Biolabs) for 25 minutes at 37° C. before stopping the reaction with EDTA at a final concentration of 125 mM. Genomic DNA enriched for fragments between 300 to 500 bp was purified using AMPure XP beads (Beckman Coulter) and eluted in water. End-repair, dA-tailing, Y-shape adaptor ligation, triple-SPRI purification and size selection, and PCR enrichment were performed as described (Thong et al., 2011). A total of 60 uniquely barcoded libraries was developed (22 for resistant plants, 8 for moderately-resistant plants, 28 for susceptible plants, and two for RG-PtoS) and subdivided into four pools of 15 libraries. Each of the four pools was sequenced in one lane by the Genomics Resources core facility at Weill Cornell Medical College using Illumina HiSeq 2500 paired-end sequencing (2×100 bp) technology. LA2109 was sequenced separately in one lane using Illumina HiSeq 2000 paired-end sequencing (2×100 bp). All genome sequence data (fastq files) are available in the Genbank SRA (Leinonen et al., 2011) as Bioproject ID: PRJNA289640.

### Genotyping and Linkage Analysis

Illumina sequence reads from F2 plants, LA2109, and PtoS were mapped to the S. lycopersicum reference genome Heinz 1706 SL2.40 (Tomato Genome Consortium, 2012) with Bowtie 2 (Langmead and Salzberg, 2012). Duplicate reads were removed with Picard (http://picard.sourceforge.net) and local realignment around indels was performed using GATK (DePristo et al., 2011). The alignments were converted to mpileup format and SNPs called with SAMtools and bcftools respectively (Li 2011, Li et al., 2009). Biallelic SNPs unique to the LA2109 parent were selected as markers. SNPs at Hardy-Weinberg equilibrium with a minimum read depth of 2, maximum read depth of 10, genotype quality score at least 10, and a quality of at least 950 were selected for further analysis using vcftools (Danecek et al., 2011). A Perl script, snp_window.pl (https://github.com/srs218/manuscripts) was developed to bin the SNPs into windows of 100,000 bp with at least 10 SNPs per window and to calculate a consensus genotype per individual per bin to result in 1,852 markers. An additional 2,681 markers were generated in 1 kb bins with at least 5 SNPs per bin around the linked region on chromosome 2. Missing genotypes were inferred by using the no double-crossover method in r/QTL (Broman et al., 2003). A linkage map was constructed using the marker physical location from S. lycopersicum Heinz 1706 SL2.40 (Tomato Genome Consortium, 2012). A single QTL genome scan using extended Haley-Knott regression was performed to identify candidate QTL loci. A two-dimensional, 2-QTL scan was performed to determine interaction and heritability. All QTL computations were performed using rQTL software (Broman et al., 2003).

High Resolution Mapping with Molecular Markers

Molecular markers in the qRph1 region were generated on the basis of sequence gaps in the LA2109 genome by comparison with the reference Heinz 1706 reference genome sequence SL2.40. These gaps represent potential DNA insertions or deletions in the LA2109 genome. Primers were designed to span these gap regions and to amplify a fragment from both LA2109 and RG-PtoS genomic DNA. DNA from two young leaves of each resistant F2 plant was isolated using an extraction buffer (200 mM Tris-HCl pH 8.0, 250 mM NaCl, 25 mM EDTA pH 8.0, 0.5% w/v SDS) and dissolved in 100 μl distilled water. 1 μl DNA was used for PCR amplification. All resistant plants were genotyped with 11 indel molecular markers and plants with a recombination event in the candidate region were used as ‘informative recombinants’ to delimit the region conferring T1 resistance.

### RNA Extraction, Reverse Transcription PCR and Quantitative RT-PCR

Leaf tissue was ground in liquid nitrogen and total RNA was extracted with TRIzol reagent following the manufacturer's instructions (Life Technologies, cat. #15596-018). The concentration of total RNA was determined with a Nanodrop spectrophotometer (Thermo Scientific Co.). RNA (lug) was treated with TURBO DNA-free™ Kit (Life Technologies, cat. AM1907M) and tested for DNA contamination by amplifying SlCBL1 (Solyc12g015870) for 25 cycles; no PCR product was detected. The RNA was then used for reverse transcription PCR (RT-PCR). First strand cDNA was synthesized following instructions provided with the RevertAid First Strand cDNA Synthesis kit (Thermo Scientific Co., cat. #K1622). 1 μl and 0.4 μl of cDNA templates were used for RT-PCR and quantitative-RT-PCR (qRT-PCR), respectively. SlCBL1 was used as an internal control for RT-PCR and as a reference gene for qRT-PCR data normalization (Pombo et al., 2014).

### Phylogenetic Analysis

Tomato genes similar to the qRph1 candidate Solyc02g072470 at the nucleotide and amino acid levels were identified from a BLAST search of the Heinz ITAG2.3 gene predictions (Tomato Genome Consortium, 2012) and used for phylogenetic analyses along with Solyc02g072470, Solyc02g070890 (SlFLS2.1) and Solyc02g070910 (SlFLS2.2). PhyML in SeaView software was used for all analyses with one hundred bootstraps under default settings of the GTR model for nucleotide analysis and the JTT model for amino acid analysis (Gouy et al, 2010). Phylogenetic trees were optimized by FigTree software (http://tree.bio.ed.ac.uk/software/figtree/).

The following example is provided to illustrate certain embodiments of the invention. It is not intended to limit the invention in any way.

### Example I

### Solanum habrochaites Accession LA2109 Exhibits Resistance to Pseudomonas syringae pv. tomato Race 1 Strain T1

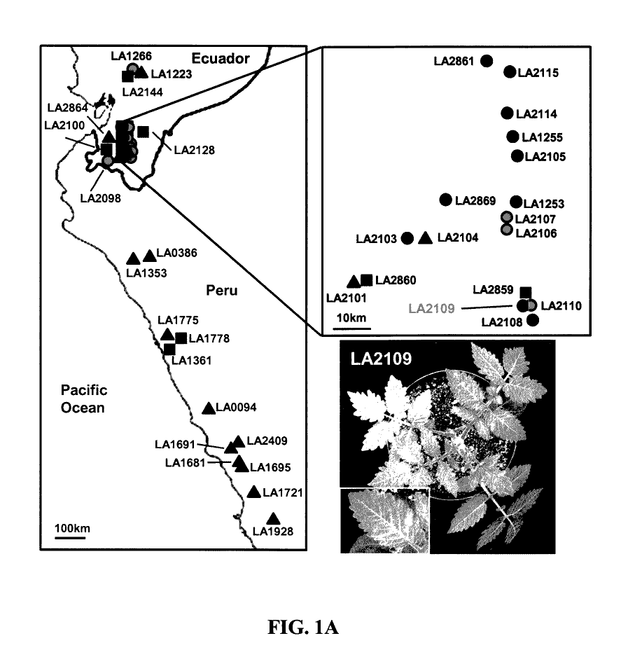

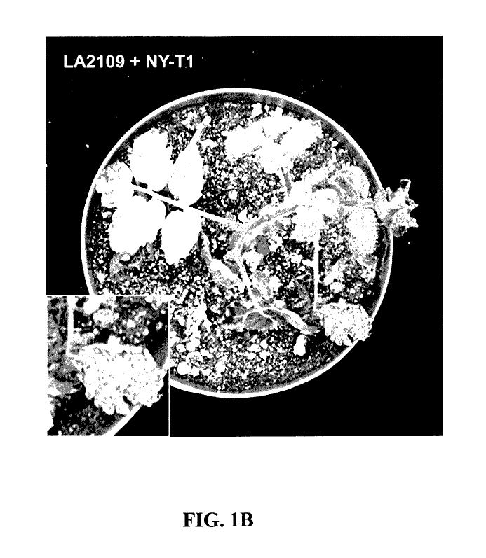

In an initial screen of germplasm from the Tomato Genetics Resource Center core collection we discovered that Solanum habrochaites accession LA2109 developed no symptoms of bacterial speck disease after inoculation with the P. s. pv. tomato (Pst) race 1 strain T1 (FIG. 1). This accession was collected in Loja province in southern Ecuador in 1980 by a team led by Charles Rick from the University of California-Davis. Photographs of the accession in its native site are available along with notes describing LA2109 as a “single very large plant in the village. Very vigorous and scrambling over a low stone wall” (tgrc.ucdavis.edu). We subsequently tested an expanded set of 35 accessions from S. habrochaites including many that were collected from the same geographical area as LA2109 (FIG. 1, Table 1, Table 2). Fourteen additional accessions were found to be resistant to T1 with 13 being from the same region (Loja province) as LA2109, indicating these accessions might share a common genetic basis for their resistance to T1. To ensure that the glabrous leaf morphology of LA2109 did not interfere with our inoculation protocol, we tested the highly virulent Pst race 1 strain NY-T1 on LA2109 (Jones et al, 2015) and found that this strain caused severe bacterial speck disease (FIG. 1B).

TABLE 1
S. habrochaites accessions that were screened for
resistance to Pseudomonas syringae pv. tomato T1.

| S. habrochaites | | Disease |
|---|---|---|
| accession¹ | Collection site, region and country | index² |
| LA0094 | Canta-Yangas, Lima, Peru | 3 |
| LA0386 | Cajamarca, Cajamarca, Peru | 3 |
| LA1223 | Alausi, Chimborazo, Ecuador | 3 |
| LA1253 | Pueblo Nuevo-Landangue, Loja, Ecuador | 1 |
| LA1255 | Loja (Predestal district), Loja, Ecuador | 1 |
| LA1266 | Pallatanga, Chimborazo, Ecuador | 1 |
| LA1353 | Contumaza, Cajamarca, Peru | 3 |
| LA1361 | Pariacoto, Ancash, Peru | 2 |
| LA1681 | Mushhka, Lima, Peru | 3 |
| LA1691 | Yauyos, Lima, Peru | 3 |
| LA1695 | Cacachhuasiin, Cacra, Lima, Peru | 3 |
| LA1721 | Ticrapo Viejo, Huancavelica, Peru | 3 |
| LA1775 | Rio Casma, Ancash, Peru | 3 |
| LA1778 | Rio Casma, Ancash, Peru | 2 |
| LA1928 | Ocana, Ayacucho, Peru | 3 |
| LA2098 | Sabiango, Loja, Ecuador | 1 |
| LA2100 | Sozorango, Loja, Ecuador | 2 |
| LA2101 | Cariamanga, Loja, Ecuador | 3 |
| LA2103 | Lansaca, Loja, Ecuador | 1 |
| LA2104 | Pena Negra, Loja, Ecuador | 3 |
| LA2105 | Jardin Botanico, Loja, Loja, Ecuador | 1 |
| LA2106 | Yambra, Loja, Ecuador | 1 |
| LA2107 | Los Loris, Loja, Ecuador | 1 |
| LA2108 | Portete de Anganuma, Loja, Ecuador | 1 |
| LA2109 | Yangana #1, Loja, Ecuador | 1 |
| LA2110 | Yangana #2, Loja, Ecuador | 1 |
| LA2114 | San Juan, Loja, Ecuador | 1 |
| LA2115 | Pucala, Loja, Ecuador | 1 |
| LA2128 | Zumbi, Zamor-Chinchipe, Ecuador | 2 |
| LA2144 | Chanchan, Chimborazo, Ecuador | 2 |
| LA2409 | Miraflores, Lima, Peru | 3 |
| LA2859 | Yangana, Loja, Ecuador | 3 |
| LA2860 | Cariamanga, Loja, Ecuador | 2 |
| LA2861 | Las Juntas, Loja, Ecuador | 1 |
| LA2864 | Sozorango, Loja, Ecuador | 3 |
| LA2869 | Matola-LA Toma, Loja, Ecuador | 1 |

¹Accessions were obtained from the Tomato Genetics Resource Center (tgrc.ucdavis.edu)
²Disease index is described in the Results section. Index ranges from 1 for no symptoms of bacterial speck disease to 3 for extensive symptoms of the disease.

TABLE 2
Geographical coordinates¹ of the collections sites for the
Solanum habrochaites accessions that were screened.

| S. habrochaites | | |
|---|---|---|
| accession | Latitude | Longitude |
| LA0094 | 11°31′0″S; decimal: −11.516667 | 76°41′0″W; decimal: −76.683333 |
| LA0386 | 7°9′36″S; decimal: −7.160000 | 78°30′0″W; decimal: −78.500000 |
| LA1223 | 2°11′45″S; decimal: −2.195900 | 78°51′2″W; decimal: −78.850600 |
| LA1253 | 4°8′24″S; decimal: −4.140000 | 79°12′0″W; decimal: −79.200000 |
| LA1255 | 3°59′50″S; decimal: −3.997222 | 79°12′36″W; decimal: −79.210000 |
| LA1266 | 2°0′40″S; decimal: −2.011111 | 78°58′30″W; decimal: −78.975000 |
| LA1353 | 7°22′0″S; decimal: −7.366667 | 78°48′0″W; decimal: −78.800000 |
| LA1361 | 9°33′0″S; decimal: −9.550000 | 77°53′30″W; decimal: −77.891667 |
| LA1681 | 12°45′0″S; decimal: −12.750000 | 75°50′0″W; decimal: −75.833333 |
| LA1691 | 12°27′35″S; decimal: −12.459722 | 75°55′5″W; decimal: −75.918056 |
| LA1695 | 12°48′45″S; decimal: −12.812500 | 75°47′1″W; decimal: −75.783611 |
| LA1721 | 13°26′7″S; decimal: −13.435278 | 75°27′55″W; decimal: −75.465278 |
| LA1775 | 9°31′0″S; decimal: −9.516667 | 77°53′0″W; decimal: −77.883333 |
| LA1778 | 9°33′S; decimal: −9.557400 | 77°42′W; decimal: −77.715000 |
| LA1928 | 14°23′55″S; decimal: −14.398611 | 74°49′22″W; decimal: −74.822778 |
| LA2098 | 4°21′57″S; decimal: −4.365833 | 79°48′46″W; decimal: −79.812778 |
| LA2100 | 4°21′36″S; decimal: −4.360000 | 79°47′47″W; decimal: −79.796389 |
| LA2101 | 4°19′56″S; decimal: −4.332222 | 79°33′45″W; decimal: −79.562500 |
| LA2103 | 4°14′S; decimal: −4.233333 | 79°27′W; decimal: −79.450000 |
| LA2104 | 4°13′41″S; decimal: −4.228056 | 79°24′16″W; decimal: −79.404444 |
| LA2105 | 4°2′3″S; decimal: −4.034167 | 79°11′56″W; decimal: −79.198889 |
| LA2106 | 4°12′S; decimal: −4.203333 | 79°13′W; decimal: −79.230000 |
| LA2107 | 4°11′S; decimal: −4.183333 | 79°13′W; decimal: −79.216667 |
| LA2108 | 4°24′25″S; decimal: −4.406944 | 79°9′33″W; decimal: −79.159167 |
| LA2109 | 4°22′8″S; decimal: −4.368889 | 79°10′35″W; decimal: −79.176389 |
| LA2110 | 4°22′1″S; decimal: −4.366944 | 79°10′31″W; decimal: −79.175278 |
| LA2114 | 3°56′0″S; decimal: −3.933333 | 79°13′30″W; decimal: −79.225000 |
| LA2115 | 3°50′29″S; decimal: −3.841389 | 79°12′43″W; decimal: −79.211944 |
| LA2128 | 3°53′37″S; decimal: −3.893600 | 78°46′49″W; decimal: −78.780200 |
| LA2144 | 2°17′24″S; decimal: −2.290000 | 78°58′57″W; decimal: −78.982500 |
| LA2409 | 12°17′22″S; decimal: −12.289444 | 75°48′27″W; decimal: −75.807500 |
| LA2859 | 4°21′47″S; decimal: −4.363000 | 79°10′29″W; decimal: −79.174600 |
| LA2860 | 4°20′0″S; decimal: −4.333333 | 79°33′0″W; decimal: −79.550000 |
| LA2861 | 3°49′0″S; decimal: −3.816667 | 79°16′0″W; decimal: −79.266667 |
| LA2864 | 4°20′0″S; decimal: −4.333333 | 79°47′0″W; decimal: −79.783333 |
| LA2869 | 4°7′S; decimal: −4.16667 | 79°22′W; decimal: −79.366667 |

¹Geographical coordinates were obtained from the Tomato Genetics Resource Center (tgrc.ucdavis.edu).

LA2109 has Both Pto-Dependent and Pto-Independent Resistance to P. syringae pv. tomato

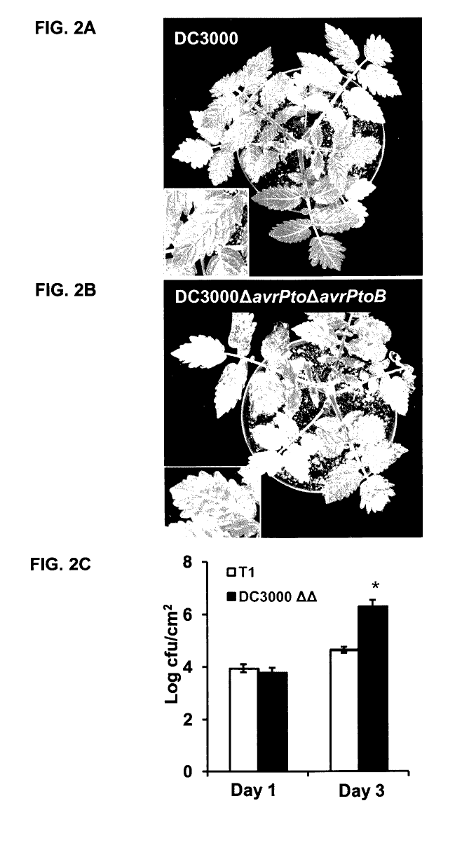

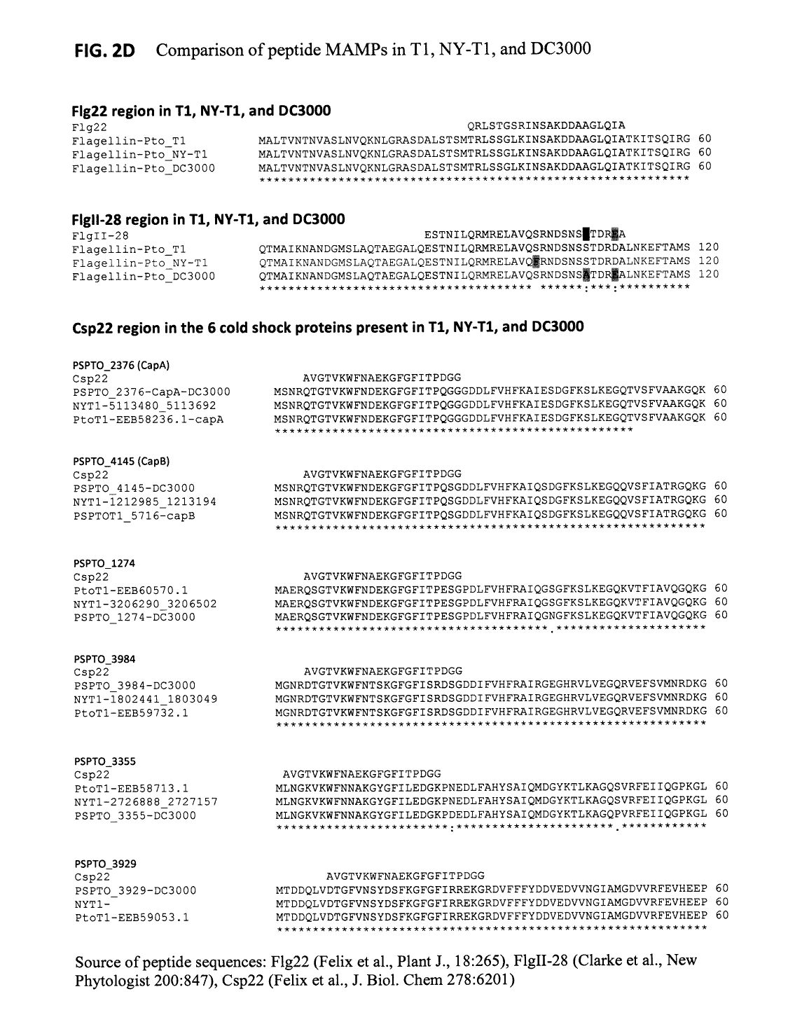

Pto-mediated resistance, which involves recognition of the effector proteins AvrPto or AvrPtoB, has been previously reported in S. habrochaites, so we tested if LA2109 also carries this gene (Riely and Martin, 2001). Upon inoculation of LA2109 with Pst DC3000, a strain which carries both avrPto and avrPtoB, we observed no signs of bacterial speck disease (FIG. 2A). However, inoculation with a mutant of DC3000 which lacks both effector genes resulted in the appearance of numerous specks and chlorosis starting after 3-4 days, indicating that LA2109 does indeed have a functional Pto gene (FIG. 2B). Consistent with these results, populations of the DC3000 mutant reached significantly higher levels than T1 in leaves of LA2109 3 days after inoculation (FIG. 2C). Like other race 1 strains, T1 is fully virulent on Pto-expressing tomato varieties (Shan et al., 2000). Together, these data suggest that LA2109 has both Pto-mediated disease resistance, and Pto-independent resistance to T1.

Resistance in LA2109 to P. syringae pv. tomato T1 does not Involve AvrPtoB or Pto

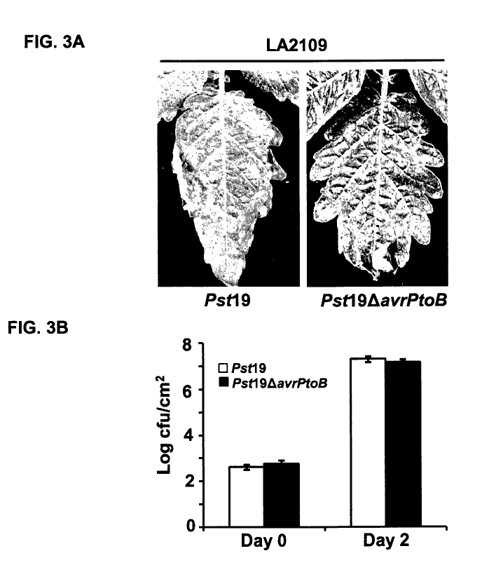

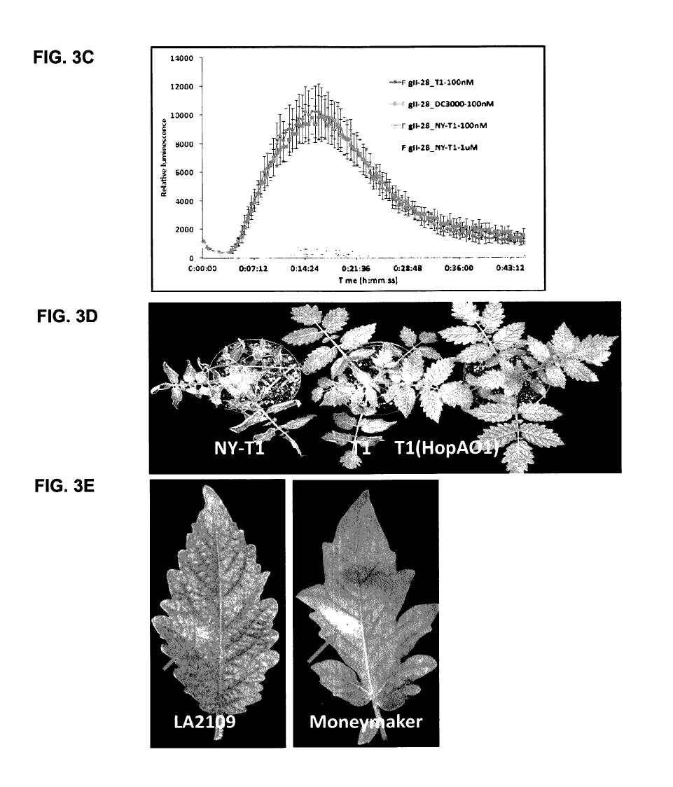

Pst T1 does not have avrPto and although it does have an avrPtoB homolog, the corresponding protein does not appear to accumulate and the strain is therefore not recognized by Pto (Kunkeaw et al., 2010, Lin et al., 2006). Nevertheless we wished to examine the possibility that LA2109 detects AvrPtoB in T1 thus explaining its resistance to this strain. A T1 strain carrying a deletion in avrPtoB is not available so we relied instead on a T1-like strain Pst19 which also has an avrPtoB gene that does not appear to be expressed; importantly an avrPtoB mutant is available for Pst19 (Kunkeaw et al., 2010). Inoculation of LA2109 with Pst19 or Pst19ΔavrPtoB resulted in similar symptoms of speck disease (FIG. 3A) and the two strains reached similar population sizes in leaves (FIG. 3B). Thus, the Pto gene in LA2109 and the avrPtoB homolog in Pst19, and likely in T1, do not contribute to the resistance to T1 that we observed in LA2109.

LA2109 Response to Known Peptide MAMPs, Type III Effectors, or Coronatine does not Appear to Play a Role in its Resistance to T1

The available genome sequences of T1, NY-T1, and DC3000 allowed us to investigate whether specific features unique to T1 might explain LA2109 resistance to this strain. The Flg22 region is identical in all three Pst strains as are the Csp22 regions in all six of the cold shock proteins (FIG. 2D), thus making it unlikely these MAMPs play a role in LA2109 resistance to T1. LA2109 was previously reported to be more responsive to Csp22 than Rio Grande-PtoS (Veluchamy et al., 2014). However, in our genetic analysis described below we found no correlation between Csp22 responsiveness and resistance to T1. This observation, combined with the lack of any difference in this MAMP among the three strains, suggests it is not relevant to LA2109 T1 resistance.

The FlgII-28 region in NY-T1 flagellin has a phenylalanine at position 16 whereas T1 has a serine (Jones et al., 2015). This substitution is known to compromise FlgII-28 PTI-elicitation activity and it could possibly explain the hyper-virulence of NY-T1 (Cai et al., 2011). However, the FlgII-28 region in strains T1 and DC3000 differs by just two amino acids and synthetic peptides of both are fully active in eliciting PTI (FIG. 2D and FIGS. 3C-E) (Cai et al., 2011). Thus it seems unlikely that the variation in FlgII-28 plays a role in the differential response of LA2109 to these three Pst strains.

We next considered the possibility that a specific type III effector in T1 is recognized by LA2109 thereby activating ETI. However, the repertoires of effectors in T1 and NY-T1 were reported recently to be identical except that NY-T1 also has an effector candidate with limited similarity to HopAO1 (Jones et al., 2015). It is possible that NY-T1 evades ETI because HopAO1 interferes with the recognition of an effector by LA2109. To test this possibility we cloned the HopAO1 candidate effector gene from NY-T1 and transformed it into T1. Inoculation of the T1(hopAO1) strain onto LA2109 revealed no change in T1 virulence (FIGS. 3C-E). Together these results suggest that LA2109 does not recognize a unique effector in T1.

Finally, we considered whether LA2109 might respond differently to coronatine as NY-T1 and DC3000 produce this phytotoxin, but T1 does not (Almeida et al., 2009; Jones et al., 2015). Infiltration of 1.25 nM of coronatine into leaves of LA2109 and Moneymaker (susceptible to T1) resulted in a similar amount of chlorosis (FIGS. 3C-E). This observation suggests that coronatine probably does not play a role in the LA2109 response to T1 and it supports an earlier report that production of coronatine does not correlate with Pst virulence (Kunkeaw et al., 2010).

Resistance of LA2109 is Effective Against Other P. syringae Pathovars

To determine if LA2109 is resistant to other strains of P. syringae, we chose a subset of diverse strains from a recent study (Cai et al., 2011) and tested them first on a Pto-expressing tomato cultivar (RG-PtoR) to determine whether they lacked avrPto and avrPtoB and were virulent on tomato.

### Development of an F2 Population to Investigate the Genetic Basis of T1 Resistance in LA2109

To further explore the resistance in LA2109, we initiated a genetic approach by crossing LA2109 to RG-PtoS (female parent), a variety fully susceptible to T1. An F1 plant, which was verified based on its morphology and later by DNA markers, successfully set seeds for an F2 population. Newly-emerged branches from the F1 and LA2109 plants were excised, rooted and grown for 6 weeks, and seedlings were inoculated with T1. Leaves of F1 plants developed a few specks seven days after inoculation, whereas no disease symptoms were observed on LA2109 (data not shown). As expected, RG-PtoS developed severe symptoms of speck disease.

### Identification of Quantitative Trait Loci on Chromosomes 2 and 8

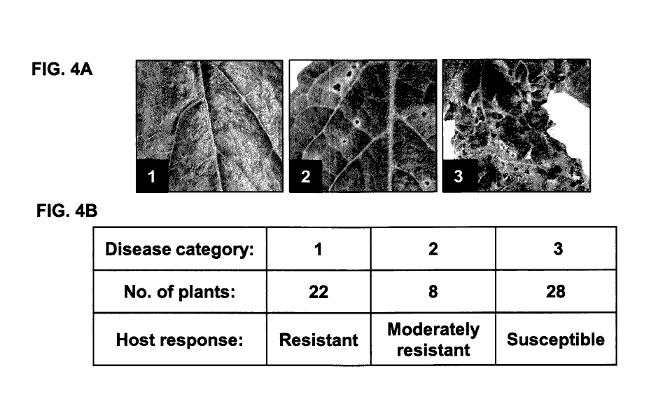

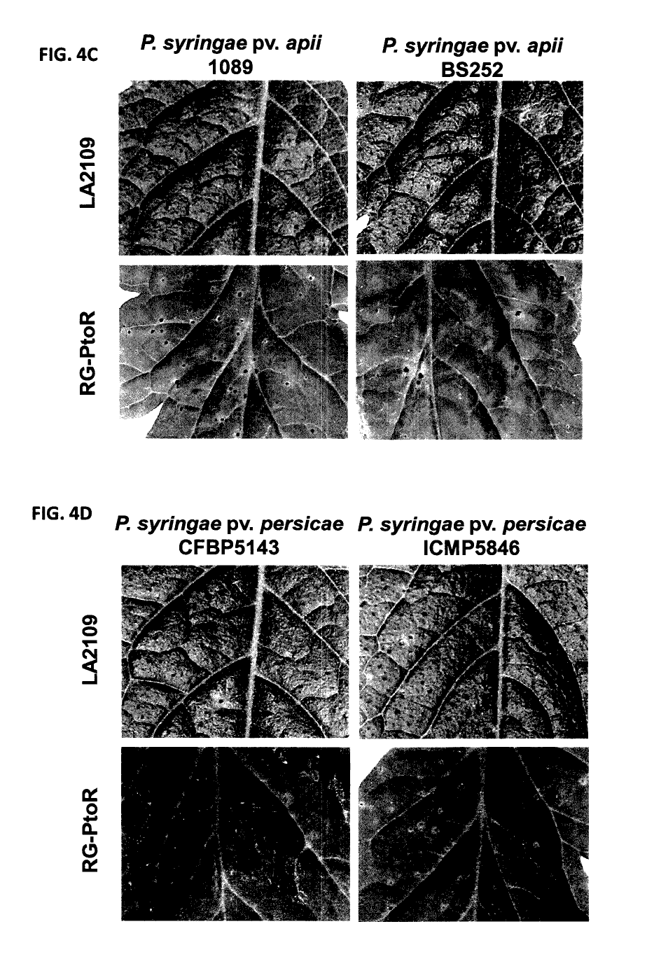

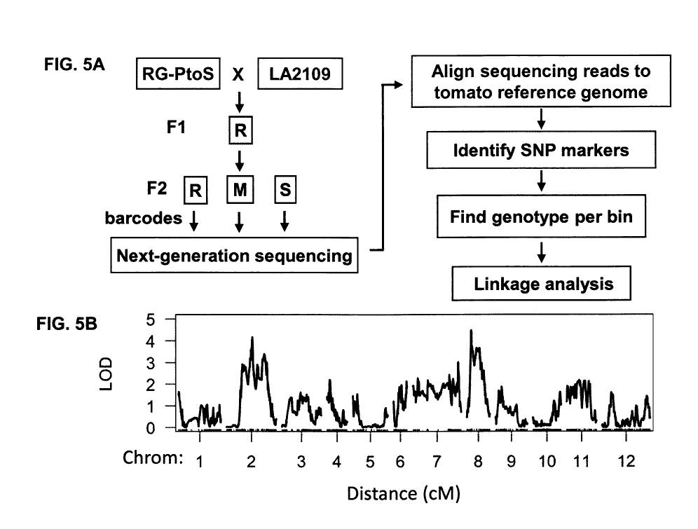

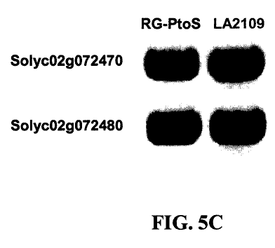

A total of 423 F2 plants along with the parents (LA2109 and RG-PtoS) were inoculated with T1 and disease symptoms were scored seven days later using a system in which LA2109, which developed no or very few specks was scored as a 1 and RG-PtoS, with many dead leaves, was scored as a 3. The F2 plants were similarly scored and classified into three groups: resistant (scored as a 1), moderately resistant (2), and susceptible (3) (FIG. 4). Plants scored as moderately-resistant encompassed a range of disease symptoms and the numbers of plants in each of the three groups did not fit any simple segregation ratio suggesting multiple loci might be involved in T1 resistance. For further analysis, 22 resistant, 8 moderately-resistant, and 28 susceptible F2 plants were identified and used for low coverage (3×) whole genome sequencing (FIG. 5A and Table 3). Parental accessions, LA2109 and RG-PtoS, were sequenced at approximately 53× and 6×, respectively (Table 3). The low coverage of the sequence data from the individual F2 plants made accurate genotyping difficult, so a consensus genotype was called in bins along the reference genome chromosome (data not shown). Linkage analysis detected major QTLs on chromosomes 2 and 8 (FIG. 5B). The QTL on chromosome 2, accounting for 23.7% of the phenotypic variation and referred to as qRph1 (quantitative resistance to Pst T1 in S. habrochaites 1), spanned genome coordinates 35,128,470 bp to 40,887,114 bp (5.8 Mbp) with a maximum LOD score of 4.2 found at 38,157,470 bp of the reference tomato genome sequence SL2.40 (Tomato Genome Consortium, 2012) (FIG. 5B). The QTL on chromosome 8, accounting for 25.9% of the phenotypic variation and referred to as qRph2, spanned genome coordinates 2,089,886 bp to 54,513,866 bp (52.4 Mbp) and had a maximum LOD score of 4.5. This QTL was not investigated further due to the lack of recombination on chromosome 8 which precluded fine mapping of the region.

Segregation distortion was detected on chromosome 1 between approximately 2.5 Mbp and 70.3 Mbp and chromosome 6 between 0 Mbp and 34.3 Mbp, where no RG-PtoS homozygotes were detected in the F2 plants. Both of these chromosomes are known to have loci involved in unilateral incompatibility, where pistils of self-incompatible species reject the pollen of self-compatible species (ui1.1, on chromosome 1 which encodes an S-locus F-box protein, maps to approximately 33.3 Mbp-49.4 Mbp, near the self-incompatibility locus (Li and Chetelat, 2015) and ui6.1, which encodes a Cullin protein, is located on chromosome 6 at 49.6 mb (Li and Chetelat, 2010).

High Resolution Mapping of qRph1

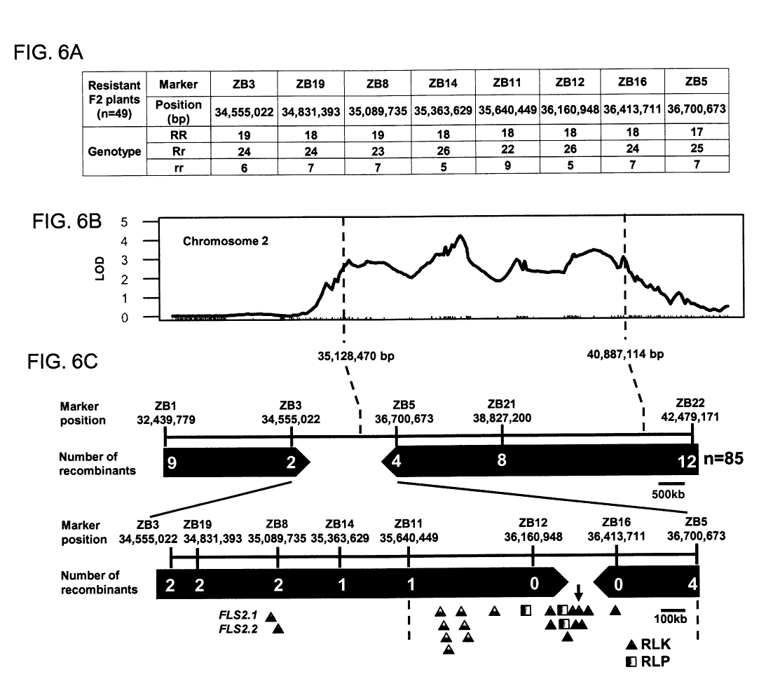

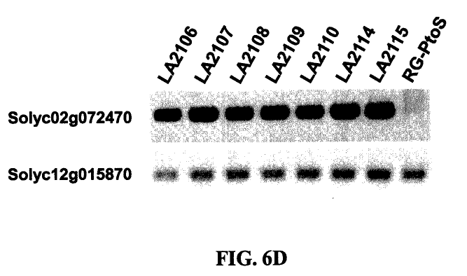

To further test whether the candidate qRph1 is associated with resistance to T1, we designed one cleaved amplified polymorphic sequence (CAPS) marker (ZB3), four co-dominant insertion/deletion (INDEL) markers (ZB19, ZB8, ZB14 and ZB5) and three markers (ZB11, ZB12 and ZB16) with a large deletion in LA2109 that need two pairs of primers to genotype both parents at the same location. We used these 8 markers to genotype 49 T1-resistant F2 plants (selected from the population of 423 F2 plants) (FIG. 6A, Table 4). If the qRph1 region confers resistance to T1 then we expected a statistically significant variance from a 1:2:1 ratio for markers closely linked to the qRph1 locus. Genotyping data indicated that, depending on the marker used, 35-39% of resistant plants were homozygous for LA2109 DNA, 45-53% were heterozygous (LA2109/RG-PtoS), and 10-18% were homozygous for RG-PtoS (FIG. 6A). Chi-square tests of a 1:2:1 ratio for each marker revealed P values of: ZB3 (p=0.03<0.05), ZB19 (p=0.08), ZB8 (p=0.05), ZB14 (p=0.03<0.05), ZB11 (p=0.15), ZB12 (p=0.03<0.05), ZB16 (p=0.08), and ZB5 (p=0.13) indicating that the DNA region associated with markers ZB3, ZB8, ZB14 and ZB12 is not segregating randomly (FIG. 6A). These data support the linkage of this region to qRph1 as identified from our QTL analysis.

To further delimit the region containing qRph1, we combined our analysis of the 49 plants above with an additional 36 T1-resistant plants derived from the 423 F2 plants (for a total of 85 plants; FIG. 6B-C). The additional plants were genotyped using our initial markers to choose plants with informative recombination breakpoints in the candidate region (FIG. 6C). Extra markers outside the candidate region (ZB1, ZB21 and ZB22) were designed to demonstrate the decline in recombination as qRph1 was approached (FIG. 6C and Table 4). FLS2.1 and FLS2.2 are located in this general region, but two recombinants excluded these genes as candidates for qRph1 (FIG. 6C). This marker-based mapping, in combination with our QTL analysis, ultimately delimited a 1,060 kb region between 35,640,449 bp (defined by ZB11) and 36,700,673 bp (defined by ZB5) as the region conferring resistance to T1 (FIG. 6C; the coordinates are based on the Heinz 1706 SL2.40 reference genome).

The Region Encompassing qRph1 has Several Potential Immunity-Related Genes Including Some Encoding RLPs and RLKs

By inspection of the tomato reference genome sequence we found that 139 genes are annotated in the 1,060 kb region defined above (Table 5). It is possible that LA2109 encodes additional genes in this region, but a high-quality S. habrochaites genome sequence is not available and our reads of LA2109 were insufficient for generating a de novo targeted assembly of this region. Among the 139 genes are some in classes which have been implicated in plant immunity, including two CBL-interacting kinases (Cipk1 and Cipk16; Cipk6 plays a role in Pst immunity, (de la Torre et al., 2013)), RPW8.2 (conferring broad-spectrum powdery mildew resistance, (Xiao et al., 2001)) as well as WRKY and bZIP transcription factors (Tsuda and Somssich, 2015).

Perhaps of greatest interest is the presence in this region of numerous genes encoding receptor-like proteins (RLPs) and receptor-like protein kinases (RLKs) (FIG. 6C and Table 5). Such proteins have well-established functions in recognition of MAMPs and subsequent immune signaling. One of the RLK genes in the region, SERK2, has been implicated in plant immunity (Chen et al., 2014, Roux et al., 2011); the annotation for the SERK2 tomato gene (Solyc02g072320), however, indicates only a 288 bp open-reading frame and for now we excluded it from further analysis. We also excluded two RLK genes for which we could not detect a transcript in leaves (Solyc02g071800 and Solyc02g071870) and two RLP genes one of which seemed incorrectly annotated (Solyc02g072380) and the other for which we could not detect a transcript in leaves (Solyc02g072390).

### One RLK-Encoding Gene is Highly Expressed in LA2109 and Other T1-Resistant Accessions but not in Rio Grande

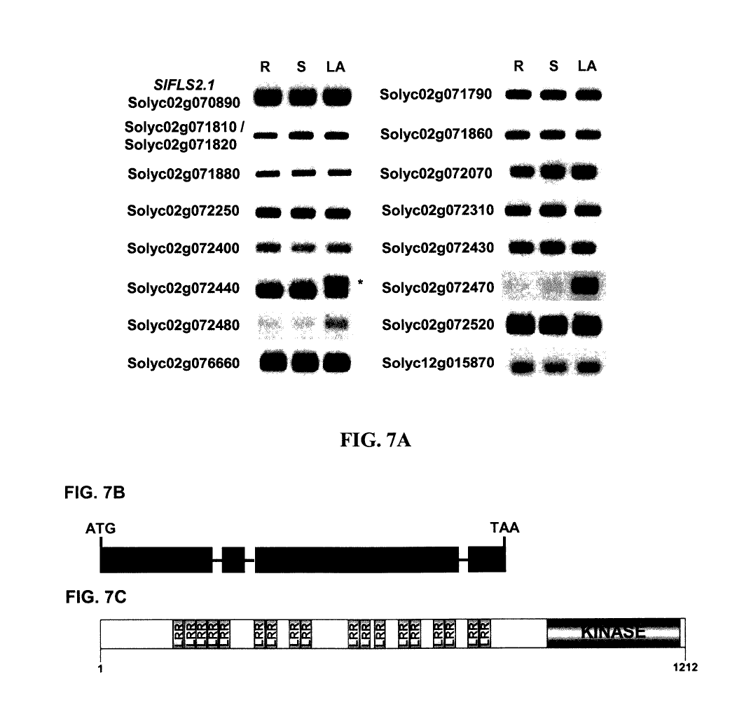

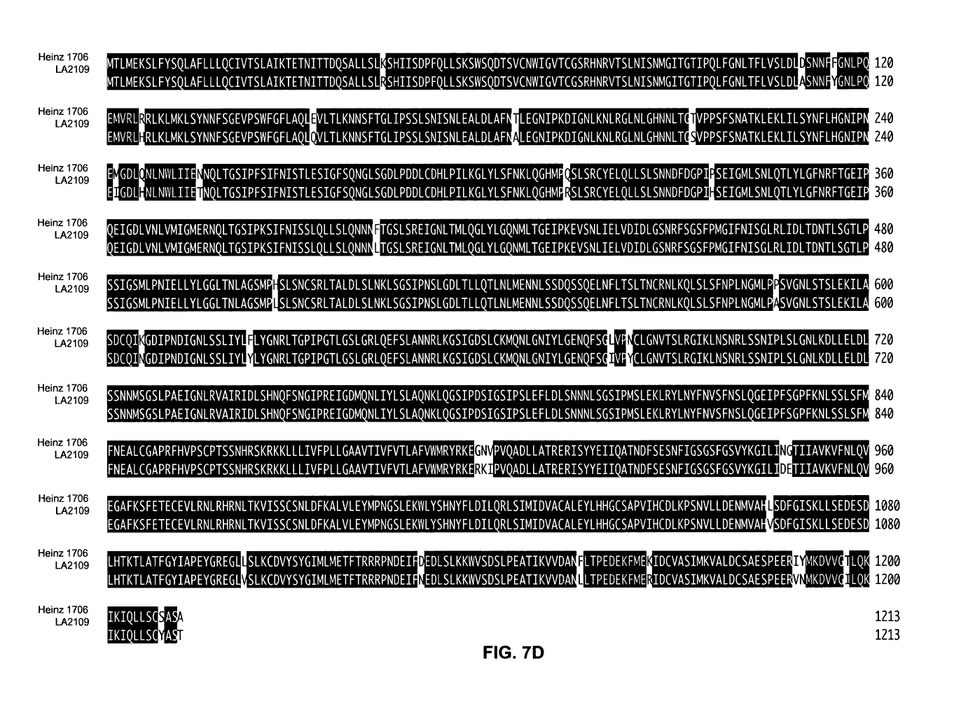

For the remaining one RLP gene and 14 RLK genes we developed primers and examined the abundance of their transcripts in leaves of LA2109 and a pair of Rio Grande lines (RG-PtoR and RG-PtoS) that are near-isogenic for the Pto region and which were expected to be identical in the 1,060 kb segment (FIG. 7A and Table 6). SlFLS2.1 and a gene encoding a calcineurin B-like protein were included as controls. After verifying there was no DNA contamination of our RNA samples, we used semi-quantitative RT-PCR to measure transcript abundance. RLK genes Solyc02g071810 and Solyc02g071820 have highly similar nucleotide sequences and we could only design primers to detect transcripts of both genes. These two genes and eleven of the other ones examined had similar transcript abundance in all three of the tomato lines (FIG. 7A). However, two, Solyc02g072470 and Solyc02g072480, had higher transcript abundance in LA2109 than in RG-PtoR and RG-PtoS. The lower transcript abundance in the latter two lines was not due to DNA differences as the primers successfully amplified the gene sequences from RG-PtoS and LA2109 genomic DNA (FIG. 5C). We focused on Solyc02g072470 for subsequent experiments because it is highly expressed in LA2109 whereas Soly02g072480 is not (FIG. 7A). Six additional S. habrochaites accessions which originate from the same area as LA2109 and are resistant to T1 were also found to have increased abundance of Solyc02g072470 transcripts.

Solyc02g072470 Transcript Abundance is Increased in Leaves Upon Treatment with MAMPs

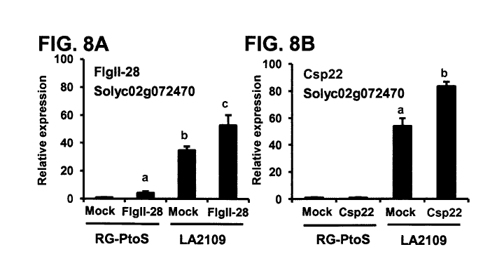

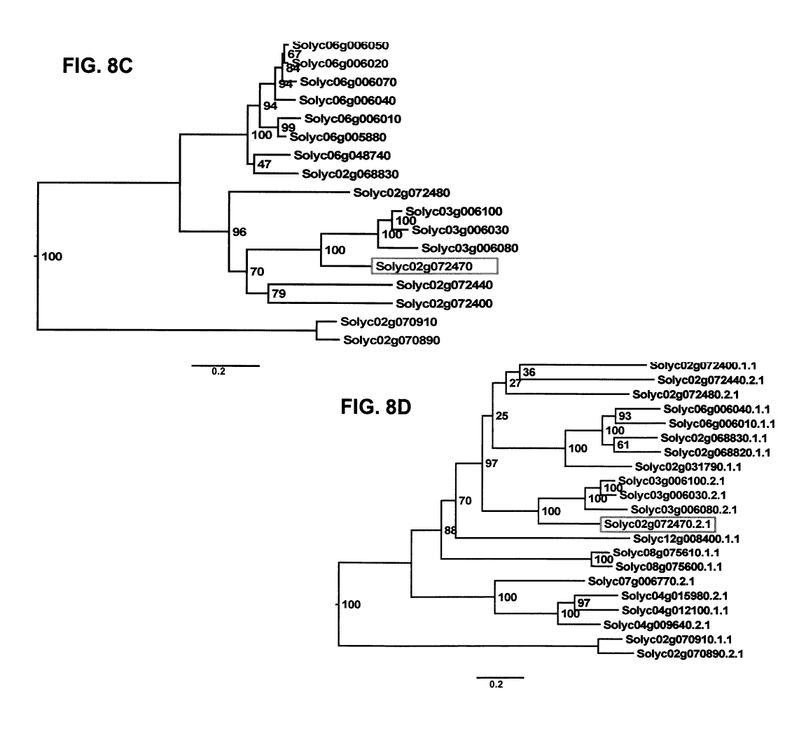

The expression of genes encoding PRRs and PTI-related proteins is often induced upon treatment with MAMPs (Rosli et al., 2013, Zipfel et al., 2006). We therefore used quantitative RT-PCR to analyze expression of Solyc02g072470 six hours after treatment of leaves with FlgII-28 (Cal et al., 2011, Clarke et al., 2013) or Csp22 (Felix and Boller, 2003). Transcript abundance of this gene was increased in response to FlgII-28 in both RG-PtoS (4-fold) and LA2109 (1.5-fold) although the transcript abundance in LA2109 was much higher in uninduced leaves compared to RG-PtoS (FIG. 8A-B). After treatment of leaves with Csp22 Solyc02g072470 was also significantly induced in LA2109; Csp22 treatment had no effect on transcript abundance in RG-PtoS.

### Solyc02g072470 has 18 Predicted LRRs and is a Class XII LRR Receptor-Like Kinase

The Solyc02g072470 gene is annotated as having four exons, and its protein is predicted to have 18 leucine-rich repeats (LRRs) and an intracellular kinase domain (FIG. 7B-D). Its predicted amino acid sequence in LA2109 is 97% identical with its ortholog in Heinz 1706. By using its DNA and protein sequences in a BLAST search, we identified 14 and 18 tomato genes, respectively, that are similar to Solyc02g072470 (FIGS. 8C-D). Like FLS2, the protein is an LRR XII RLK although its amino acid sequence is very different from that PRR and it has been placed in a distinct clade that contains several of the closely-related genes we examined in FIG. 7A (Sakamoto et al., 2012).

### DISCUSSION

We initially discovered that S. habrochaites accession LA2109 showed no signs of speck disease after being inoculated with Pst T1 and subsequently found that such resistance is common among S. habrochaites accessions collected in a localized region in southern Ecuador. This finding raised the possibility that these accessions have a common genetic basis for this phenotype. To investigate the underlying mechanism involved, we focused on LA2109 as representative of these accessions and found that it also expressed a Pto-like activity. This was not unexpected as another S. habrochaites accession and other wild relatives of tomato have been reported to have Pto (Riely and Martin, 2001, Rose et al., 2005).

We observed that LA2109 is susceptible to two other sequenced Pst strains (NY-T1 and DC3000ΔavrPtoΔavrPtoB) but a comparison of factors involved in host interactions among these three strains did not reveal any obvious explanations for the differential responses. This prompted us to use a mapping-by-sequencing approach that ultimately defined QTLs on chromosomes 2 (qRph1) and 8 (qRph2) that are associated with T1 resistance. Recombination suppression on chromosome 8 impeded the ability to further delineate the boundaries of qRph2. The region containing qRph1 on chromosome 2 was further delimited by molecular markers and found to contain 139 genes. One gene provides a promising candidate for qRph1 as it is highly expressed in LA2109 and other T1-resistant S. habrochaites accessions, but not in RG-PtoS.

Our consideration of whether specific MAMPs might potentially explain the differential resistance of LA2109 to T1, DC3000ΔavrPtoΔavrPtoB, and NY-T1 did not produce any likely candidates among the three known peptide MAMPs of Pst. It remains possible that another MAMP present in T1, but missing from the other two strains, is recognized by LA2109 and activates a strong PTI response. Consistent with this hypothesis, the essentially identical repertoire of type III effectors in T1 and NY-T1 suggests ETI is not involved in LA2109 resistance to T1. It is possible that amino acid differences in one or more effectors in T1 compared to NY-T1 allows it/them to be detected by LA2109. However, the lack of any R protein-like encoding genes in our candidate region also suggests ETI is not involved in the phenotype. Finally, we considered the possible involvement in LA2109 resistance of coronatine which is produced by NY-T1 and DC3000, but not by T1. However, we saw no difference in the sensitivity to this phytotoxin between LA2109 and another tomato cultivar susceptible to T1. A previous study also reported that production of coronatine does not correlate with Pst virulence (Kunkeaw et al., 2010). Collectively, the known differences among these three Pst strains did not suggest any factor that might explain the specific resistance in LA2109 to T1.

We discovered that although LA2109 is susceptible to NY-T1 and DC3000ΔavrPtoΔavrPtoB, it is resistant to four diverse P. syringae pathovars (pathogens of celery and peach) which are able to cause disease on the Pto-expressing tomato line RG-PtoR. To date, we have identified just three strains that lack avrPto and avrPtoB and cause disease on LA2109-DC3000ΔavrPtoΔavrPtoB, NY-T1 and Pst19 (a genome sequence is not available for Pst19).

Our initial analysis to understand the genetic basis of LA2109 resistance indicated that the phenotype was not segregating in a simple fashion and might be quantitative. By using a ‘mapping-by-sequencing’ approach involving low coverage whole genome sequencing of F2 plants and higher coverage data from the parental accessions, we were able to identify two QTLs and, in the case of qRph1, identify markers to define candidate loci for conferring resistance. Strict filtering of F2 SNPs was necessary mainly due to divergence between the two parental accessions which resulted in uncertainty in the true mapping location of many reads derived from the LA2109 parent. While low-coverage data can not easily be used to accurately determine genotype at individual loci, by pooling loci and calling a consensus genotype for linked loci in the F2 population, we were able to make genotype calls.

Despite the complications due to low sequence coverage in the F2 plants, by using additional DNA marker-based mapping in a large F2 population, we were able to delimit a 1,060 kb region that is associated with resistance to T1. The genome sequence surrounding and including this region contains many genes encoding receptor-like kinase (RLKs), including the two FLS2 genes present in tomato (FLS2.1 and FLS2.2). We observed no difference in the transcript abundance of FLS2.1 in LA2109 and RG-PtoS and data from our QTL analysis and high-resolution mapping placed both genes outside of the region conferring T1 resistance. Within the 1,060 kb region 139 genes have been annotated in the most recent version of the tomato gene predictions (ITAG release SL2.4; (Fernandez-Pozo et al., 2015)). None of these genes encode NB-LRR proteins although there are several other genes which belong to classes that are implicated in defense responses.

We chose to investigate the genes encoding RLKs/RLPs in the 1,060 kb region because of the well-known role of these types of proteins in PTI. Our initial intention was to determine if some of these genes are not expressed in leaves so as to exclude them from being qRph1. However, of the 18 genes we examined, a transcript was detected for 15 (Solyc02g071800, Solyc02g07870 and Solyc02g072390 were not expressed in leaves). Interestingly, four of the RLK genes appear to be paralogs; they are clustered within a 57 kb region and likely arose by duplication events (Solyc02g072400, Solyc02g072440, Solyc02g072470, and Solyc02g072480). Two of these genes, Solyc02g072470 and Solyc02g072480, had higher transcript abundance in LA2109 than in RG-PtoS. Solyc02g072470 is especially striking in this regard and we found its transcript is also highly abundant in the other T1-resistant accessions that were collected near LA2109. Furthermore, we found that transcript abundance of Solyc02g072470 was increased in leaves exposed to FlgII-28 or Csp22. Such induced expression is a characteristic of genes which encode PRRs (Rosli et al., 2013). A recent study reported that LA2109 has a higher ROS response to Csp22 compared to RG-PtoS (Veluchamy et al., 2014). However, in an analysis of 139 RG-PtoS x LA2109 F2 plants we saw no correlation between response to Csp22 and resistance to T1 (R²=0.01274).

A screen of 278 accessions of tomato wild species for resistance to two race 1 Pst strains (A9 and 407) was reported recently (Thapa et al., 2015). Five accessions were resistant to these strains with two of them being from S. habrochaites (LA2869 and LA1777). Using a series of LA1777 introgression lines four QTLs were identified as contributing to A9 resistance, each of which explained 10-12% of the phenotypic variation. Of potential interest is one of these QTLs, bsRr1-2, located on chromosome 2 at position 95 cM of the genetic map for the LA1777 population. Although it is not possible to determine precisely how this position corresponds to the tomato reference genome coordinates, based on the positions of the markers used in the LA1777 map it appears that bsRr1-2 lies about 8,100 kb away from the 1,060 kb region we have identified. Nevertheless, it is possible that our locus and the ones described in that study contribute to a common pathway enhancing resistance to race 1 Pst strains. Regardless of whether they contribute to the same pathway or to different mechanisms, the introgression into tomato varieties of the qRph1 region and the four LA1777 QTLs has the potential to provide some level of resistance to the increasingly prevalent race 1 strains of Pst. In addition, in light of the fact that LA2109 is resistant to some diverse P. syringae pathovars, our data indicate that qRph1 provides protection to a broader range of bacterial pathogens as has been reported for EFR and Xa21 (Lacombe et al., 2010; Mendes et al., 2010; Tripathi et al., 2014).

TABLE 3
Illumina read mapping metrics. Data for parents and F2 individuals. R =
resistant F2, MR =- medium resistant F2, S = susceptible F2

| | | Fragments | | Mapped | |
|---|---|---|---|---|---|
| | | After | Properly- | in | |
| Sample | Total | Duplicate | paired | proper | Coverage |
| Name | Fragments | Removal | Fragments | pair (%) | Depth (x) |
| LA2109 | 212860803 | 207326783 | 130190120 | 61.2 | |
| PtoS | 23601150 | 22331615 | 21509003 | 91.1 | 5.8 |
| MR11 | 11470186 | 10986580 | 8354653 | 72.8 | 2.8 |
| MR122 | 10616173 | 10289488 | 8404054 | 79.2 | 2.7 |
| MR137 | 11692754 | 11324178 | 8302771 | 71.0 | 2.9 |
| MR18 | 10976104 | 10353930 | 8111152 | 73.9 | 2.7 |
| MR207 | 14785662 | 14286880 | 10907819 | 73.8 | 3.7 |
| MR30 | 11976041 | 11606780 | 8834166 | 73.8 | 3.0 |
| MR31 | 11734673 | 11224585 | 8824365 | 75.2 | 2.9 |
| MR77 | 15483798 | 14823042 | 11671463 | 75.4 | 3.8 |
| R100 | 10134809 | 9858146 | 6880893 | 67.9 | 2.5 |
| R111 | 10759498 | 10605887 | 7594228 | 70.6 | 2.7 |
| R112 | 10694082 | 10451856 | 8012251 | 74.9 | 2.7 |
| R132 | 9826729 | 9584503 | 6755256 | 68.7 | 2.5 |
| R149 | 10382194 | 9982260 | 7723191 | 74.4 | 2.6 |
| R15 | 9276070 | 8874826 | 6941617 | 74.8 | 2.3 |
| R155 | 12092987 | 11643784 | 8358687 | 69.1 | 3.0 |
| R156 | 11328913 | 11048121 | 8058760 | 71.1 | 2.9 |
| R17 | 11274233 | 10833868 | 7740262 | 68.7 | 2.8 |
| R19 | 14271142 | 13868982 | 9952024 | 69.7 | 3.6 |
| R198 | 12066307 | 11625942 | 8883321 | 73.6 | 3.0 |
| R202 | 12539971 | 12103014 | 8426395 | 67.2 | 3.1 |
| R215 | 10526919 | 10096309 | 7958825 | 75.6 | 2.6 |
| R221 | 10301328 | 9993732 | 7370328 | 71.5 | 2.6 |
| R222 | 10265270 | 9935362 | 7171611 | 69.9 | 2.6 |
| R225 | 11566639 | 11291279 | 7745370 | 67.0 | 2.9 |
| R47 | 13032836 | 12570891 | 9184205 | 70.5 | 3.2 |
| R49 | 11216377 | 10707244 | 8143672 | 72.6 | 2.8 |
| R59 | 10213359 | 9894728 | 7235657 | 70.8 | 2.6 |
| R6 | 10968639 | 10460746 | 8171071 | 74.5 | 2.7 |
| R64 | 12105419 | 11038845 | 8933151 | 73.8 | 2.9 |
| R8 | 11370645 | 10871947 | 8363901 | 73.6 | 2.8 |
| S1 | 10092947 | 9857648 | 7028058 | 69.6 | 2.5 |
| S110 | 15378221 | 15000266 | 11785876 | 76.6 | 3.9 |
| S116 | 12421933 | 12025389 | 9151256 | 73.7 | 3.1 |
| S130 | 13726544 | 13217699 | 10092209 | 73.5 | 3.4 |
| S131 | 13081800 | 12591614 | 9990173 | 76.4 | 3.3 |
| S133 | 12974887 | 12509301 | 9294120 | 71.6 | 3.2 |
| S135 | 12420298 | 12289756 | 9105888 | 73.3 | 3.2 |
| S136 | 11709736 | 11545439 | 9286403 | 79.3 | 3.0 |
| S139 | 11914648 | 11603624 | 8610644 | 72.3 | 3.0 |
| S159 | 12072324 | 11733383 | 8443649 | 69.9 | 3.0 |
| S16 | 8625015 | 8456381 | 6026048 | 69.9 | 2.2 |
| S181 | 10349455 | 10061335 | 7583821 | 73.3 | 2.6 |
| S183 | 14269559 | 13670370 | 10176089 | 71.3 | 3.5 |
| S184 | 12874712 | 12318363 | 10360622 | 80.5 | 3.2 |
| S193 | 13293565 | 12884042 | 9734696 | 73.2 | 3.3 |
| S213 | 12218691 | 11760996 | 8548655 | 70.0 | 3.0 |
| S26 | 9734480 | 9495351 | 7398605 | 76.0 | 2.5 |
| S28 | 13758629 | 13200149 | 10569696 | 76.8 | 3.4 |
| S36 | 16795027 | 16037982 | 11951111 | 71.2 | 4.1 |
| S39 | 7911537 | 7709725 | 5712976 | 72.2 | 2.0 |
| S43 | 13145628 | 12720574 | 9807838 | 74.6 | 3.3 |
| S46 | 14678630 | 14313268.5 | 11394002 | 77.6 | 3.7 |
| S55 | 13629556 | 13188313 | 9271526 | 68.0 | 3.4 |
| S73 | 13016185 | 12602900.5 | 10130267 | 77.8 | 3.3 |
| S83 | 13283243 | 12955998.5 | 10022596 | 75.5 | 3.3 |
| S88 | 9990048 | 9756807.5 | | | 2.5 |
| S89 | 11996453 | 11679334 | 9072422 | 75.6 | 3.0 |
| S90 | 11501884 | 12591638 | 8587735 | 74.7 | 3.3 |

| | | |
|---|---|---|
| Average for F2 plants | 73.0 | 3.0 |

TABLE 4
Molecular markers used for the high-resolution mapping of Rph1

| Marker | Primer location | Primers | RG-PtoS | LA2109 |
|---|---|---|---|---|
| ZB1 | SL2.40ch02:32439779 | CAGACCCAAATACAGTTGTAGATG | 363 bp | 243 bp |
| | SL2.40ch02:32440140 | CTAGCAAATTATATCATGACATTCG | | |
| ZB3 | SL2.40ch02:34555022 | GAATTGGCTTACTAAAGTGTTTGG | Digested by HinfI, | Not digested by |
| | SL2.40ch02:34555443 | CAAGCAAAGCAACCTCATTCAACTCC | 197 bp & 225 bp | HinfI, 422 bp |
| ZB19 | SL2.40ch02:34831393 | AGCCAACTTTGGTTGAAGGTG | 230 bp | 130 bp |
| | SL2.40ch02:34831605 | CTCCTTAGGGTAGAGATTCGCC | | |
| ZB8 | SL2.40ch02:35089735 | AATGTGGCATCGCACGAAGCAC | 383 bp | 156 bp |
| | SL2.40ch02:35090118 | CATTATCCATTGGGAATTTCTCC | | |
| ZB14 | SL2.40ch02:35363629 | CTTTCTATTAATCTCTTCTCCCCCA | 500 bp | 250 bp |
| | SL2.40ch02:35364150 | ACTATGGAATGGTTAGTGGAACCT | | |
| ZB11-1 | SL2.40ch02:35640449 | GATTTGGTTGCCTGAAAGTTATTC | x | 250 bp |
| | SL2.40ch02:35641543 | CTCTTTGTATATAATTCTTTGAGAT | | |
| ZB11-2 | SL2.40ch02:35640674 | GGCTCAATCCCGAAGTGGTT | 512 bp | x |
| | SL2.40ch02:35641161 | TCCAAGTCTTATTGGACAACTTTCT | | |
| ZB12-1 | SL2.40ch02:36160948 | GTTTTTGGGTTTATTATGATCATTG | x | 250 bp |
| | SL2.40ch02:36161444 | GTGTCAAGAACACTGGTTGCA | | |
| ZB12-2 | SL2.40ch02:36160948 | GTTTTTGGGTTTATTATGATCATTG | 500 bp | x |
| | SL2.40ch02:36161948 | CGTAGGTTATGGGTTGCTCGAGTC | | |
| ZB16-1 | SL2.40ch02:36413739 | TTTTGAGTGCACTTGCTCCT | x | 234 bp |
| | SL2.40ch02:36414522 | GTCACTAGATGCTAAAGAAGGGC | | |
| ZB16-2 | SL2.40ch02:36413739 | TTTTGAGTGCACTTGCTCCT | 442 bp | x |
| | SL2.40ch02:36414160 | ACTGCGTTAGTTGGGAGAGAG | | |
| ZB5 | SL2.40ch02:36700673 | GTGAAGTCTCGAGAATATATTTGTC | 554 bp | 361 bp |
| | SL2.40ch02:36701226 | GGCAAGAGGAACATGTTCCGTAAGG | | |
| ZB21-1 | SL2.40ch02:38827214 | TGAGTTGTCGACGTCTATGATG | 696 bp | 222 bp |
| | SL2.40ch02:38827889 | AGGATACTTGAGCAAAAGGCT | | |
| ZB21-2 | SL2.40ch02:38827214 | TGAGTTGTCGACGTCTATGATG | 378 bp | x |
| | SL2.40ch02:38827568 | ACGTCCCAATTCCCATAAATTACT | | |
| ZB22 | SL2.40ch02:42479171 | CCTTTTCAATTACCCTCGCTGG | 450 bp | 222 bp |
| | SL2.40ch02:42479799 | AAGCTTTGTAACTCCAAGTATGTTT | | |

*x indicates no fragment amplified from this accession with these primers. For ZB3, digestion of the 422 bp PCR product with HinfI produces 197 bp & 225 bp fragments from RG-PtoS; the 422 bp product from LA2109 is not digested by this enzyme. Sequences are SEQ ID Nos: 43-72, from top to bottom.

TABLE 6
Primers for the determination of transcript
abundance in FIG. 7

| Primer designation | Nucleotide sequence |
|---|---|
| FLS 2.1 | AATGGGAACTTGGACAACA (3)* |
| Solyc02g070890-F: | |
| FLS2.1 | ACACCAAAGCTGAATACATCTAC (4) |
| Solyc02g070890-R: | |
| Solyc02g071790-F: | ATGGTTTCAATCGATAAGTTGTAC (5) |
| Solyc02g071790-R: | CCGACTTCTTGAGGTATTCTCCCG (6) |
| Solyc02g071800-F: | ATGGGTCTTCAATGCATGTGTTG (7) |
| Solyc02g071800-R: | TAGGTCCAGTTCCTCTAGCTGAG (8) |
| Solyc02g071810/ | TACACCGTGACACTGCACTTTGC (9) |
| Solyc02g071820-F: | |
| Solyc02g071810/ | GGGCTTGAAATTAGCTTCAAAAG (10) |
| Solyc02g071820-R: | |
| Solyc02g071860-F: | ATGCCAAGAATCTTCAATTTC (11) |
| Solyc02g071860-R: | ATCCTGCCCTATGAGAGATAT (12) |
| Solyc02g071870-F: | ATGTTGCCACTAAAAAGAGCTG (13) |
| Solyc02g071870-R: | AGGAAGATTAATACCCTTAAGAG (14) |
| Solyc02g071880-F: | ATGTATCCAACCATAGCTTGG (15) |
| Solyc02g071880-R: | GGGAAGCTTTACCAATTCAGGTGGG |
| | (16) |
| Solyc02g072070-F: | TGGATCATTGTGCTCCTTGAGT (17) |
| Solyc02g072070-R: | CCAGCTCAAGGTCAGTCCAG (18) |
| Solyc02g072250-F: | TGGCTCCATTGTTCCTCTCA (19) |
| Solyc02g072250-R: | TGAACAGGAGAAACAGGGCA (20) |
| Solyc02g072310-F: | CATTGGGTGGCCACTACTCC (21) |
| Solyc02g072310-R: | TTCAGCAGGAATCGGACCAG (22) |
| Solyc02g072390-F: | ACAACTTGGCAACCTCTCCT (23) |
| Solyc02g072390-R: | GTTGTTGCCGAGTGCAAGATT (24) |
| Solyc02g072400-F: | GCAACTTGCTTAGCCATGAAT (25) |
| Solyc02g072400-R: | CGCAAACGAGAAAACTCAGGT (26) |
| Solyc02g072430-F: | CTTTGTTCAGACTGGGGAGAT (27) |
| Solyc02g072430-R: | TCACTTCTTTCCAAGACTCCGA (28)_ |
| Solyc02g072440-F: | ACCACGCAGCATATCCAACT (29) |
| Solyc02g072440-R: | GAGCTGGGTATAGATCCACCAA (30) |
| Solyc02g072470-F: | GGCAATCTGCCTCAAGAAATGG (31) |
| Solyc02g072470-R: | GGATGGAGGAACAGTACCAGT (32) |
| Solyc02g072480-F: | TGTCAAGCAACTGGACCTCA (33) |
| Solyc02g072480-R: | AGTGCTGGAGTTCTGGCAAA (34) |
| Solyc02g072520-F: | ACCTCCACTTGAATTCATCTACTCT |
| | (35) |
| Solyc02g072520-R: | CCATGAGACTATGCCAGTGAGG (36) |
| Solyc02g076660-F: | GGGGGCAGATGGAACACTTA (37) |
| Solyc02g076660-R: | AGCACCAAAAAGACGGTTTTCA (38) |
| Solyc12g015870-F: | CCATCCAAATGCTCCGATCGATGA |
| | (39) |
| Solyc12g015870-R: | TGCCTCTCAATGAAGCCTTGTTGC |
| | (40) |
| Solyc02g072470qRT-F: | TGATCAGTCCGCGCTTCTTT (41) |
| Solyc02g072470qRT-R: | GTGCCTAGAGCCACAAGTGA (42) |

*Numbers in parentheses are SEQ ID NOs.

### REFERENCES

Almeida, N. F., S. Yan, M. Lindeberg, D. J. Studholme, D. J. Schneider, B. Condon, et al. 2009. A draft genome sequence of Pseudomonas syringae pv. tomato T1 reveals a type III effector repertoire significantly divergent from that of Pseudomonas syringae pv. tomato DC3000. Mol Plant Microbe Interact 22: 52-62. doi:10.1094/MPMI-22-1-0052.
Anderson, D. M. and D. W. Frank. 2012. Five mechanisms of manipulation by bacterial effectors: a ubiquitous theme. PLoS pathogens 8: e1002823. doi:10.1371/journal.ppat.1002823.
Arredondo, C. R. and R. M. Davis. 2000. First report of Pseudomonas syringae pv. tomato race 1 on tomato in California. Plant Disease 84: 371.
Boller, T. and G. Felix. 2009. A renaissance of elicitors: perception of microbe-associated molecular patterns and danger signals by pattern-recognition receptors. Annu Rev Plant Biol 60: 379-406.
Broman, K. W., H. Wu, S. Sen and G. A. Churchill. 2003. R/qtl: QTL mapping in experimental crosses. Bioinformatics 19: 889-890.
Buell, C. R., V. Joardar, M. Lindeberg, J. Selengut, I. T. Paulsen, M. L. Gwinn, et al. 2003. The complete genome sequence of the Arabidopsis and tomato pathogen Pseudomonas syringae pv. tomato DC3000. Proc Natl Acad Sci USA 100: 10181-10186.
Buonaurio, R., V. M. Stravato and C. Capelli. 1996. Occurence of Pseudomonas syringae pv. tomato race 1 in Italy on Pto genebearing tomato plants. Phytopathology 144: 437-440.
Cai, R., J. Lewis, S. Yan, H. Liu, C. R. Clarke, F. Campanile, et al. 2011. The plant pathogen Pseudomonas syringae pv. tomato is genetically monomorphic and under strong selection to evade tomato immunity. PLoS Pathog 7: e1002130.
Cai, R., S. Yan, H. Liu, S. Leman and B. A. Vinatzer. 2011. Reconstructing host range evolution of bacterial plant pathogens using Pseudomonas syringae pv. tomato and its close relatives as a model. Infect Genet Evol. doi:10.1016/j.meegid.2011.07.012.
Cai, R. M., J. Lewis, S. C. Yan, H. J. Liu, C. R. Clarke, F. Campanile, et al. 2011. The plant pathogen Pseudomonas syringae pv. tomato is genetically monomorphic and under strong selection to evade tomato immunity. PLoS Pathog 7: e1002130.
Chakravarthy, S., A. C. Velasquez and G. B. Martin. 2009. Assay for pathogen-associated molecular pattern (PAMP)-triggered immunity (PTI) in plants. J Visualiz Exper: http://www.jove.com/index/Details.stp?ID=1442.
Chen, X., S. Zuo, B. Schwessinger, M. Chern, P. E. Canlas, D. Ruan, et al. 2014. An XA21-associated kinase (OsSERK2) regulates immunity mediated by the XA21 and XA3 immune receptors. Molecular plant 7: 874-892. doi:10.1093/mp/ssu003.
Cheng, W., K. R. Munkvold, H. Gao, J. Mathieu, S. Schwizer, S. Wang, et al. 2011. Structural analysis of Pseudomonas syringae AvrPtoB bound to host BAK1 reveals two similar kinase-interacting domains in a type III effector. Cell Host & Microbe 10: 616-626. doi:10.1016/j.chom.2011.10.013.
Chinchilla, D., C. Zipfel, S. Robatzek, B. Kemmerling, T. Nurnberger, J. D. Jones, et al. 2007. A flagellin-induced complex of the receptor FLS2 and BAK1 initiates plant defence. Nature 448: 497-500.
Clarke, C. R., D. Chinchilla, S. R. Hind, F. Taguchi, R. Miki, Y. Ichinose, et al. 2013. Allelic variation in two distinct Pseudomonas syringae flagellin epitopes modulates the strength of plant immune responses but not bacterial motility. New Phytol 200: 847-860. doi:10.1111/nph.12408.
Danecek, P., A. Auton, G. Abecasis, C. A. Albers, E. Banks, M. A. DePristo, et al. 2011. The variant call format and VCFtools. Bioinformatics 27: 2156-2158. doi:10.1093/bioinformatics/btr330.
de la Torre, F., E. Gutierrez-Beltran, Y. Pareja-Jaime, S. Chakravarthy, G. B. Martin and O. del Pozo. 2013. The tomato calcium sensor 01110 and its interacting protein kinase Cipk6 define a signaling pathway in plant immunity. The Plant cell 25: 2748-2764. doi:10.1105/tpc.113.113530.
DePristo, M. A., E. Banks, R. Poplin, K. V. Garimella, J. R. Maguire, C. Hartl, et al. 2011. A framework for variation discovery and genotyping using next-generation DNA sequencing data. Nature genetics 43: 491-498. doi:10.1038/ng.806.
Dodds, P. N. and J. P. Rathjen. 2010. Plant immunity: towards an integrated view of plant-pathogen interactions. Nat Rev Genet 11: 539-548.
Dou, D. and J. M. Zhou. 2012. Phytopathogen effectors subverting host immunity: different foes, similar battleground. Cell Host Microbe 12: 484-495. doi:10.1016/j.chom.2012.09.003.
Felix, G. and T. Boller. 2003. Molecular sensing of bacteria in plants. The highly conserved RNA-binding motif RNP-1 of bacterial cold shock proteins is recognized as an elicitor signal in tobacco. J Biol Chem 278: 6201-6208.
Fernandez-Pozo, N., N. Menda, J. D. Edwards, S. Saha, I. Y. Tecle, S. R. Strickler, et al. 2015. The Sol Genomics Network (SGN)-from genotype to phenotype to breeding. Nucleic acids research 43: D1036-1041. doi:10.1093/nar/gku1195.
Goldman, A. 2008. The heirloom tomato: From garden to table: receipes, portraits, and history of the world's most beautiful fruit. First ed. Bloomsbury, USA.
Gouy, M., S. Guindon and O. Gascuel. 2010. SeaView version 4: A multiplatform graphical user interface for sequence alignment and phylogenetic tree building. Mol Biol Evol 27: 221-224.
Grandillo, S., R. T. Chetelat, S. Knapp, D. Spooner, I. Peralta, M. Cammareri, et al. 2011. Solanum sect. Lycopersicon. In: C. Kole, editor Wild Crop Relatives: Genomics and Breeding Resources, Vegetables. Springer-Verlag, Berlin/Heidleberg. p. 129-215.
Guo, M., F. Tian, Y. Wamboldt and J. R. Alfano. 2009. The majority of the type III effector inventory of Pseudomonas syringae pv. tomato DC3000 can suppress plant immunity. Mol Plant Microbe Interact 22: 1069-1080. doi:10.1094/MPMI-22-9-1069.
Heese, A., D. R. Hann, S. Gimenez-Ibanez, A. M. Jones, K. He, J. Li, et al. 2007. The receptor-like kinase SERK3/BAK1 is a central regulator of innate immunity in plants. Proc Natl Acad Sci USA 104: 12217-12222.
Hind, S. R., S. R. Strickler, P. C. Boyle, Z. Bao, I. M. O'Doherty, J. A. Baccile, et al. 201x. Tomato receptor FLAGELLIN-SENSING 3 perceives flagellin and activates immune signaling. Submitted.
Jones, J. B. 1991. Bacterial speck. In: J. B. Jones, J. P. Jones, R. E. Stall and T. A. Zitter, editors, Compendium of tomato diseases. APS Press, St. Paul, Minn. p. 26-27.
Jones, L. A., S. Saha, A. Collmer, C. D. Smart and M. Lindeberg. 2015. Genome-assisted development of a diagnostic protocol for distinguishing high virulence Pseudomonas syringae pv. tomato strains. Plant Disease http://dx.doi.org/10.1094/PDIS-08-14-0833-RE.
Kunkeaw, S., S. Tan and G. Coaker. 2010. Molecular and evolutionary analyses of Pseudomonas syringae pv. tomato race 1. Mol Plant-Microbe Interact 23: 415-424.
Lacombe, S., A. Rougon-Cardoso, E. Sherwood, N. Peeters, D. Dahlbeck, H. P. van Esse, et al. 2010. Interfamily transfer of a plant pattern-recognition receptor confers broad-spectrum bacterial resistance. Nature biotechnology 28: 365-369.
Langmead, B. and S. L. Salzberg. 2012. Fast gapped-read alignment with Bowtie 2. Nat Methods 9: 357-359. doi:10.1038/nmeth.1923.
Leinonen, R., H. Sugawara, M. Shumway and C. International Nucleotide Sequence Database. 2011. The sequence read archive. Nucleic acids research 39: D19-21. doi:10.1093/nar/gkq1019.
Li, H. 2011. A statistical framework for SNP calling, mutation discovery, association mapping and population genetical parameter estimation from sequencing data. Bioinformatics 27: 2987-2993. doi:10.1093/bioinformatics/btr509.
Li, H., B. Handsaker, A. Wysoker, T. Fennell, J. Ruan, N. Homer, et al. 2009. The Sequence Alignment/Map format and SAMtools. Bioinformatics 25: 2078-2079. doi:10.1093/bioinformatics/btp352.
Li, W. and R. T. Chetelat. 2010. A pollen factor linking inter- and intraspecific pollen rejection in tomato. Science 330: 1827-1830. doi:10.1126/science.1197908.
Li, W. and R. T. Chetelat. 2015. Unilateral incompatibility gene ui1.1 encodes an S-locus F-box protein expressed in pollen of Solanum species. Proceedings of the National Academy of Sciences of the United States of America 112: 4417-4422. doi:10.1073/pnas.1423301112.
Lin, N. C., R. B. Abramovitch, Y. J. Kim and G. B. Martin. 2006. Diverse AvrPtoB homologs from several Pseudomonas syringae pathovars elicit Pto-dependent resistance and have similar virulence activities. Appl Environ Microbiol 72: 702-712.
Lin, N. C. and G. B. Martin. 2005. An avrPto/avrPtoB mutant of Pseudomonas syringae pv. tomato DC3000 does not elicit Pto-mediated resistance and is less virulent on tomato. Mol Plant Microbe Interact 18: 43-51.
Lin, N. C. and G. B. Martin. 2007. Pto- and Prf-mediated recognition of AvrPto and AvrPtoB restricts the ability of diverse Pseudomonas syringae pathovars to infect tomato. Mol Plant Microbe Interact 20: 806-815. doi:10.1094/MPMI-20-7-0806.
Maekawa, T., T. A. Kufer and P. Schulze-Lefert. 2011. NLR functions in plant and animal immune systems: so far and yet so close. Nature Immunol 12: 817-826. doi:10.1038/ni.2083.
Male, C. J. 1999. 100 Heirloom Tomatoes for the American Garden.Workman Publishing, New York.
Martin, G. B. 2012. Suppression and activation of the plant immune system by Pseudomonas syringae effectors AvrPto and AvrPtoB. In: F. Martin and S. Kamoun, editors, Effectors in Plant-Microbe Interactions. Wiley-Blackwell. p. 123-154.
Mendes, B. M. J., S. C. Cardoso, R. L. Boscariol-Camargo, R. B. Cruza, F. A. A. Mourao Filho and A. Bergamin Filho. 2010. Reduction in susceptibility to Xanthomonas axonopodis pv. citri in transgenic Citrus sinensis expressing the rice Xa21 gene. Plant Pathology 59: 68-75.
Nguyen, H. P., S. Chakravarthy, A. C. Velasquez, H. S. McLane, L. Zeng, D.-W. Park, et al. 2010. Methods to study PAMP-triggered immunity using tomato and Nicotiana benthamiana. Mol Plant-Microbe Interact 23: 991-999.
Oh, C. S. and G. B. Martin. 2011. Effector-triggered immunity mediated by the Pto kinase. Trends Plant Sci 16: 132-140.
Pedley, K. F. and G. B. Martin. 2003. Molecular basis of Pto-mediated resistance to bacterial speck disease in tomato. Annu Rev Phytopathol 41: 215-243.
Peralta, I. E., S. Knapp and D. M. Spooner. 2006. Nomenclature for wild and cultivated tomatoes. TGR Report 56: 6-12.
Pombo, M. A., Y. Zheng, N. Fernandez-Pozo, D. M. Dunham, Z. Fei and G. B. Martin. 2014. Transcriptomic analysis reveals tomato genes whose expression is induced specifically during effector-triggered immunity and identifies the Epk1 protein kinase which is required for the host response to three bacterial effector proteins. Genome Biol 15: 492. doi:10.1186/PREACCEPT-1479195265134573.
Riely, B. K. and G. B. Martin. 2001. Ancient origin of pathogen recognition specificity conferred by the tomato disease resistance gene Pto. Proc Natl Acad Sci USA 98: 2059-2064.
Robatzek, S. and L. Wirthmueller. 2013. Mapping FLS2 function to structure: LRRs, kinase and its working bits. Protoplasma 250: 671-681. doi:10.1007/s00709-012-0459-6.
Rose, L. E., C. H. Langley, A. J. Bernal and R. W. Michelmore. 2005. Natural variation in the Pto pathogen resistance gene within species of wild tomato (Lycopersicon). I. Functional analysis of Pto alleles. Genetics 171: 345-357.
Rosebrock, T. R., L. Zeng, J. J. Brady, R. B. Abramovitch, F. Xiao and G. B. Martin. 2007. A bacterial E3 ubiquitin ligase targets a host protein kinase to disrupt plant immunity. Nature 448: 370-374.
Rosli, H. G., Y. Zheng, M. A. Pombo, S. Zhong, A. Bombarely, Z. Fei, et al. 2013. Transcriptomics-based screen for genes induced by flagellin and repressed by pathogen effectors identifies a cell wall-associated kinase involved in plant immunity. Genome biology 14: R139. doi:10.1186/gb-2013-14-12-r139.
Roux, M., B. Schwessinger, C. Albrecht, D. Chinchilla, A. Jones, N. Holton, et al. 2011. The Arabidopsis leucine-rich repeat receptor-like kinases BAK1/SERK3 and BKK1/SERK4 are required for innate immunity to hemibiotrophic and biotrophic pathogens. The Plant cell 23: 2440-2455. doi:10.1105/tpc.111.084301.
Sakamoto, T., M. Deguchi, O. J. Brustolini, A. A. Santos, F. F. Silva and E. P. Fontes. 2012. The tomato RLK superfamily: phylogeny and functional predictions about the role of the LRRII-RLK subfamily in antiviral defense. BMC Plant Biol 12: 229. doi:10.1186/1471-2229-12-229.
Salmeron, J. M., G. E. D. Oldroyd, C. M. T. Rommens, S. R. Scofield, H.-S. Kim, D. T. Lavelle, et al. 1996. Tomato Prf is a member of the leucine-rich repeat class of plant disease resistance genes and lies embedded within the Pto kinase gene cluster. Cell 86: 123-133.
unknown. 1996. Tomato improvement for bacterial disease resistance for the tropics: A contemporary basis and future prospects. Proceedings, 1st International Symposium on Tropical Tomato Diseases, Recife, Brazil. Amer. Soc. Hort. Sci., Alexandria, Va.
Shan, L., P. He, J. Li, A. Heese, S. C. Peck, T. Nurnberger, et al. 2008. Bacterial effectors target the common signaling partner BAK1 to disrupt multiple MAMP receptor-signaling complexes and impede plant immunity. Cell Host & Microbe 4: 17-27.
Shan, L., P. He, J.-M. Zhou and X. Tang. 2000. A cluster of mutations disrupt the avirulence but not the virulence function of AvrPto. Mol Plant-Microbe Interact 13: 592-598.
Stamova, L., N. Bogatsevska and M. Yordanov. 1990. Resistance to race 1 of Pseudomonas syringae pv. tomato. Tomato Genet Coop Rep 40: 33.
Thapa, S. P., E. M. Miyao, R. Michael Davis and G. Coaker. 2015. Identification of QTLs controlling resistance to Pseudomonas syringae pv. tomato race 1 strains from the wild tomato, Solanum habrochaites LA1777. TAG. Theoretical and applied genetics. Theoretische and angewandte Genetik DOI 10.1007/s00122-015-2463-7. doi:10.1007/s00122-015-2463-7.
Tomato Genome Consortium. 2012. The tomato genome sequence provides insights into fleshy fruit evolution. Nature 485: 635-641.
Tripathi, J. N., J. Lorenzen, O. Bahar, P. Ronald and L. Tripathi. 2014. Transgenic expression of the rice Xa21 pattern-recognition receptor in banana (Musa sp.) confers resistance to Xanthomonas campestris pv. musacearum. Plant Biotechnol J 12: 663-673. doi:10.1111/pbi.12170.
Tsuda, K. and I. E. Somssich. 2015. Transcriptional networks in plant immunity. The New phytologist. doi:10.1111/nph.13286.
Velasquez, A. C. and G. B. Martin. 2013. Molecular mechanisms involved in the interaction between tomato and Pseudomonas syrinage pv. tomato. In: G. Sessa, editor Molecular Plant Immunity. John Wiley & Sons, Oxford, England.
Veluchamy, S., S. R. Hind, D. M. Dunham, G. B. Martin and D. R. Panthee. 2014. Natural variation for responsiveness to flg22, flgII-28, and csp22 and Pseudomonas syringae pv. tomato in heirloom tomatoes. PLoS One 9: e106119. doi:10.1371/journal.pone.0106119.
Wei, H. L., S. Chakravarthy, J. Mathieu, T. C. Helmann, P. Stodghill, B. Swingle, et al. 2015. Pseudomonas syringae pv. tomato DC3000 Type III Secretion Effector Polymutants Reveal an Interplay between HopAD1 and AvrPtoB. Cell Host Microbe 17: 752-762. doi:10.1016/j.chom.2015.05.007.
Xiang, T., N. Zong, Y. Zou, Y. Wu, J. Zhang, W. Xing, et al. 2008. Pseudomonas syringae effector AvrPto blocks innate immunity by targeting receptor kinases. Curr Biol 18: 74-80.
Xiao, S., S. Ellwood, O. Calis, E. Patrick, T. Li, M. Coleman, et al. 2001. Broad-spectrum mildew resistance in Arabidopsis thaliana mediated by RPW8. Science 291: 118-120.
Xing, W., Y. Zou, Q. Liu, J. Liu, X. Luo, Q. Huang, et al. 2007. The structural basis for activation of plant immunity by bacterial effector protein AvrPto. Nature 449: 243-247.
Young, J. M., D. W. Dye and J. P. Wilkie 1986. Bacterial Speck. In: A. F. Sherf, editor Vegetable Diseases and Their Control. J. Wiley and Sons, Inc, New York. p. 610-614.
Zhang, L. P., A. Khan, D. Nino-Liu and M. R. Foolad. 2002. A molecular linkage map of tomato displaying chromosomal locations of resistance gene analogs based on a Lycopersicon esculentum x Lycopersicon hirsutum cross. Genome/National Research Council Canada=Genome/Conseil national de recherches Canada 45: 133-146.
Zhong, S., J. G. Joung, Y. Zheng, Y. R. Chen, B. Liu, Y. Shao, et al. 2011. High-throughput illumina strand-specific RNA sequencing library preparation. Cold Spring Harb Protoc 2011: 940-949. doi:10.1101/pdb.prot5652.
Zipfel, C. 2014. Plant pattern-recognition receptors. Trends in immunology 35: 345-351. doi:10.1016/j.it.2014.05.004.
Zipfel, C., G. Kunze, D. Chinchilla, A. Caniard, J. D. Jones, T. Boller, et al. 2006. Perception of the bacterial PAMP EF-Tu by the receptor EFR restricts Agrobacterium-mediated transformation. Cell 125: 749-760.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for producing a plant exhibiting increased pathogen resistance comprising,
a) introducing a qRph1 nucleic acid into a recipient plant cell, said nucleic acid encoding at least one protein useful for conferring resistance to at least one plant pathogen, said cell exhibiting increased pathogen resistance when compared to wild type plant cells lacking said qRph1 nucleic acid.

2. The method of claim 1, wherein said plant pathogen is Pseudomonas syringae pv tomato (Pst), and said cell is a tomato plant cell.

3. A transgenic tomato plant produced by the method of claim 1.

4. The method of claim 1, wherein said at least one protein is an RLK encoded by Soly02g072470.

5. A vector comprising an RLK encoded by Soly02g072470.

6. A host cell comprising the vector of claim 5.

7. A plant comprising the cell of claim 6, said plant exhibiting increased resistance to Pst relative to plants lacking said vector.
